(19) United States
(12) Patent Application Publication
(10) Pub. No.: US 2024/0262989 A1
(43) Pub. Date: Aug. 8, 2024

(54) CONFIGURABLE HYDROGEL MATERIAL AND METHOD FOR CONFIGURING HYDROGEL MATERIALS FOR SEQUESTERING AND/OR RELEASING BIOACTIVE SUBSTANCES

(71) Applicant: LEIBNIZ-INSTITUT FÜR POLYMERFORSCHUNG DRESDEN E.V., 01069 Dresden (DE)

(72) Inventors: Passant Atallah, Dresden (DE); UWE FREUDENBERG, Dresden (DE); CARSTEN WERNER, Dresden (DE); ANNA SIEDEL, Dresden (DE); NICHOLAS DENNISON, Dresden (DE)

(73) Assignee: LEIBNIZ-INSTITUT FÜR POLYMERFORSCHUNG DRESDEN E.V., 01069 Dresden (DE)

(21) Appl. No.: 18/039,183

(22) PCT Filed: Nov. 11, 2021
(86) PCT No.: PCT/DE2021/100903

§ 371 Date: May 26, 2023

(30) Foreign Application Priority Data
Nov. 27, 2020 (DE) 10 2020 131 536.8

(51) Int. Cl. C08L 5/10; A61L 27/52; C08L 33/02

(52) U.S. Cl. CPC C08L 5/10; A61L 27/52; C08L 33/02

(57) ABSTRACT

A configurable hydrogel material for sequestration of bioactive substances in the hydrogel material and/or release of bioactive substances from the hydrogel material is disclosed. The invention further relates to a method for determining and making available a configuration for a hydrogel material for sequestration and/or release of bioactive substances.

The invention relates to a configurable hydrogel material for sequestering bioactive substances into the hydrogel material and/or releasing bioactive substances from the hydrogel material. The invention further relates to a method for determining and providing a configuration for a hydrogel material for sequestering and/or releasing bioactive substances.

Due to their biocompatibility for many applications and tissue-like mechanical properties, hydrogels are widely used both in medicine and in biotechnology, and are also used as substance absorption systems or substance release systems. Hydrogels are by definition highly hydrated, covalently or physically crosslinked polymers. In addition to the control of various biologically relevant signals such as stiffness, water content and structural convertibility and the presentation of covalently bound cell adhesion proteins or peptides, hydrogels make it possible to reversibly bind bioactive substances via non-covalent interactions and thus either to bind substances from biofluids or living tissues, i.e. to sequester them in the hydrogel, or to release substances from the hydrogels to the environment. Such bioactive substances can be protein-based signal molecules, such as, for example, cytokines, chemokines, hormones, neurotransmitters, growth factors or enzymes, which fulfill one or more biological functions and can reversibly bind to the affinity-mediating components of the hydrogel on the basis of charge interactions and/or further interactions, such as, for example, hydrophobic interactions or water bridge bonds. In addition, non-protein-based bioactive chemical substances, active substances or “small molecules” are also known, which can likewise take over the abovementioned cell-instructive signaling properties of the cytokines, chemokines, hormones, neurotransmitters or growth factors, and cause further biological effects and are reversibly bound and released by hydrogels. Furthermore, the bioactive substances can be pharmaceutical active substances. All the above-mentioned bioactive substances have a molecular weight of less than or equal to 70 kDa as an overlapping feature and can therefore penetrate into the meshes of the hydrogel networks according to the invention in a charge-dependent manner on account of their molecular size and can thus be sequestered or released therefrom. For the sake of simplicity, such bioactive substances are referred to below as substances. The prior art, for example WO 2018/162009 A2, discloses hydrogel systems in which the control of the sequestration or the release of substances and thus their affinity to them are described via the global and local charge density of the hydrogel networks. A disadvantage of the known solutions is a low specificity of the hydrogel networks for binding or releasing certain bioactive substances, so that a controlled release or binding of such substances cannot be realized to the desired extent.

Although it is possible to adapt or configure hydrogel networks in terms of their composition specifically for the substance, the development of substance-specific hydrogels is associated with an unjustifiable economic effort, given the number of existing or conceivable substance structures and the associated required number of experimental tests. A possibility is therefore requested to provide hydrogels with specific physicochemical properties for the differentiated absorption of substances from a biofluid or living tissue and/or for the differentiated release of substances into a biofluid or living tissue.

The object of the invention is thus to propose a configurable hydrogel material which can be configured with respect to a desired affinity for certain bioactive substances. It is further an object of the invention to provide a method which makes it easier to determine a configuration of a hydrogel material suitable for sequestration and/or release of certain bioactive substances.

The object is achieved by a configurable hydrogel material with the features according to claim 1 and a method according to claim 16. Further developments of the invention are indicated in the respective dependent claims.

The invention comprises a first hydrogel material which is based on at least three building blocks carrying nucleophilic groups and anionically charged under physiological conditions, preferably building blocks carrying sulfate, sulfonate, phosphate or carboxyl groups, and uncharged building blocks which have at least two electrophilic groups suitable for reaction with the nucleophilic groups, wherein the charged and uncharged building blocks are crosslinked to a polymer network, obtainable by reaction of the nucleophilic and the electrophilic groups. The composition of the hydrogel material can be configured on the basis of three parameters defining the anionic components, selected from a group of parameters P0, P1, P2, P3, wherein parameter P0 corresponds to a value from the number of ionized anionic groups, assuming 30% ionization of all anionic groups, per unit volume of the hydrogel material swollen under physiological conditions, parameter P1 corresponds to a value from the number of strongly anionic groups, with an intrinsic pKₛ value of less than 2.5, per unit volume of the hydrogel material swollen under physiological conditions, parameter P2 corresponds to a value from the number of strongly anionic groups, with an intrinsic pKₛ value of less than 2.5, per repeat unit divided by the molar mass of the repeat unit, and parameter P3 corresponds to a value for describing the amphiphilia of the anionic building blocks.

The polymer network formed from anionically charged building blocks and uncharged building blocks is an anionically charged polymer network. The anionically charged polymer network can be configured on the basis of parameters which define the anionically charged building blocks.

Furthermore, the invention comprises a second hydrogel material, which is based on charged building blocks in the form of poly(acrylic acid-co-4-acrylamidomethylbenzenesulfonic acid) and/or poly(acrylic acid-co-acrylamidoethanesulfonic acid) and/or poly(acrylic acid-co-acrylamidoethane hydrogen sulfate) and uncharged building blocks in the form of polymers or crosslinker molecules containing amino or thiol groups with at least two amino or thiol groups, wherein the charged and the uncharged building blocks are crosslinked to a polymer network, obtainable through the activation of the carboxyl groups of the poly(acrylic acid-co-4-acrylamidomethylbenzenesulfonic acid) and/or the poly(acrylic acid-co-acrylamidoethanesulfonic acid) and/or the poly(acrylic acid-co-acrylamidoethane hydrogen sulfate) with EDC/Sulfo-NHS and either a direct crosslinking with the polymers or the crosslinker molecules containing amino groups with the at least two amino groups in each case under amide formation or a functionalization of the activated carboxyl groups by means of bifunctional crosslinker molecules which in each case contain an amino group and a group capable of a Michael type addition, and the subsequent crosslinking with the polymers or the crosslinker molecules containing thiol groups with the at least two thiol groups in each case via a Michael type addition. The composition of the hydrogel material can be configured on the basis of three parameters defining the building blocks carrying the charged groups, selected from a group of parameters P0, P1, P2, P3. Parameter P0 corresponds to a value from the number of ionized anionic groups, assuming 30% ionization of all anionic groups, per unit volume of the hydrogel material swollen under physiological conditions, parameter P1 corresponds to a value from the number of strongly anionic groups, with an intrinsic pKₛ value of less than 2.5, per unit volume of the hydrogel material swollen under physiological conditions, parameter P2 corresponds to a value from the number of strongly anionic groups, with an intrinsic pKₛ value of less than 2.5, per repeat unit divided by the molecular mass of the repeat unit and parameter P3 corresponds to a value for describing the amphiphilia of the molecular structure surrounding the anionic groups.

For the purposes of the invention, biofluids or tissues are understood as synonyms for biologically relevant compartments into which the bioactive substances are released or from which the substances are sequestered.

For the purposes of the invention, bioactive substances are understood to be protein-based and non-protein-based bioactive substances, active substances and “small molecules” which have signal properties of cytokines, chemokines, hormones, neurotransmitters or growth factors and cause further biological effects. Bioactive substances can be, in particular, pharmaceutical active ingredients. A molecular weight of less than or equal to 70 kDa is considered to be the overarching feature for all above-mentioned bioactive substances.

The configurable hydrogel materials according to the invention are provided for the sequestration of substances into the hydrogel material and depletion of the substances in a biofluid or tissue and/or release of substances from the hydrogel material into the biofluid or tissue and depletion of the substances in the hydrogel material.

Anionic building block is understood as meaning a polymeric or oligomeric hydrogel building block which carries several anionic and at least 3 nucleophilic groups.

Parameters P0 to P3 are defined in more detail on the basis of the following definitions and formation rules: Parameter P0: the value for P0, which can be indicated in μmol/ml, corresponds to 30% of the total number of anionic groups based on the hydrogel volume swollen under physiological conditions (0.154 mmol/l NaCl, pH buffered to 7.4). The calculation can be calculated, for example, from the polymer concentration of the hydrogel building blocks with swelling in physiological solution.

Parameter P1: the value for P1, which can be indicated in μmol/ml, corresponds to the number of strongly anionic groups which have a pKs value of less than 2.5, based on the hydrogel volume swollen under physiological conditions (0.154 mmol/l NaCl, pH buffered to 7.4).

Parameter P2: the value for P2, which can be indicated in mmol/(g/mol), corresponds to the number of strongly anionic groups with a pKs value of less than 2.5 per anionically charged building block divided by the respective molecular weight of the anionic building block.

Parameter P3: Parameter P3, which describes the amphiphilia of the anionic building blocks, is calculated by dividing the distribution coefficient octanol/water (LogP value) of the anionic building block by the surface of the anionic building block accessible to the solvent water. This can be done as follows with the help of the ChemDraw19.0 and ChemAxon MarvinSketch 19.21 software: Using the software ChemDraw19.0, each anionic building block with a length of the polymer residue of 22 carbon atoms or, in the case of sugar-based structures with a total of 2 disaccharide units, is represented as a complete chemical structure. By using the software application ChemAxon MarvinSketch 19.21, the distribution coefficient octanol/water (LogP values) is then calculated by reading in the structural formulas represented by ChemDraw 19.0 and the surface accessible by the solvent water is calculated with a solvent radius of 1.4 Å. The value obtained is multiplied by a factor of 1000 to obtain the unit 10⁻³×[1/A² or A⁻²]. In the second hydrogel material according to the invention, it can be provided that the group capable of Michael type addition is selected from maleimide, vinyl sulfone or acrylate groups.

Furthermore, in the second hydrogel material according to the invention, it can be provided that the polymers containing amine and thiol groups are selected as uncharged building blocks from the class of polyethylene glycols (PEG), poly(2-oxazolines) (POX), polyvinylpyrrolidones (PVP), polyvinyl alcohols (PVA) and/or polyarylamides (PAM), and that the crosslinker molecules containing amine or thiol groups are non-polymeric, bifunctional crosslinker molecules.

For the first hydrogel material according to the invention and the second hydrogel material according to the invention it may be provided that poly(acrylic acid-co-4-acrylamidomethylbenzenesulfonic acid) with variable molar ratios of acrylic acid to 4-acrylamidomethylbenzenesulfonic acid in the range from 9:1 to 1:9 and molar masses in the range from 5,000 to 100,000 g/mol is selected as a charged building block, and/or poly(acrylic acid-co-acrylamidoethanesulfonic acid) with variable molar ratios of acrylic acid to acrylamidoethanesulfonic acid in the range from 9:1 to 1:9 and molar masses in the range from 5,000 to 100,000 g/mol is selected as a charged building block, and/or poly(acrylic acid-co-acrylamidoethane hydrogen sulfate) with variable molar ratios of acrylic acid to acrylamidoethane hydrogen sulfate in the range from 9:1 to 1:9 and molar masses in the range from 5,000 to 100,000 g/mol is selected as a charged building block.

Polymers with conjugated enzymatically cleavable peptides which have either lysine or cysteine as reactive amino acid in the peptide sequence can be used as uncharged building blocks for polymer network formation. In this connection, it may further be provided that the enzymatically cleavable peptides can be cleaved by human or bacterial proteases, in particular MMPs, cathepsins, elastases, aureolysin and/or blood coagulation enzymes.

According to a further development of the second hydrogel according to the invention, bioactive and/or antiadhesive molecules with an amino or carboxyl group and/or cell-instructive peptides can be attached to the hydrogel network via lysine or cysteine in the sequence on the charged building blocks poly(acrylic acid-co-4-acrylamidomethylbenzenesulfonic acid) and/or poly(acrylic acid-co-acrylamidoethanesulfonic acid) and/or poly(acrylic acid-co-acrylamidoethanesulfonic acid) or on their derivatives with groups capable of Michael type addition, forming a covalent bond. According to this further development, the bioactive molecules can be antimicrobial substances, for example antibiotics or antiseptics, or pharmaceutical active ingredients. Further, the antiadhesive molecules may be polyethylene glycols (PEG) or poly(2-oxazolines) (POX).

Further with respect to the above further development of the second hydrogel according to the invention, it may be provided that the cell-instructive peptides are peptides derived from structural and functional proteins of the extracellular matrix, such as, for example, from collagen, laminin, tenascin, fibronectin and vitronectin. The bioactive and/or antiadhesive molecules and/or cell-instructive peptides can be covalently coupled to the hydrogel networks via enzymatically cleavable peptide sequences.

Both hydrogel materials according to the invention can have a storage module of preferably 0.2 kPa to 22 kPa.

The invention further comprises a configurable physically crosslinked hydrogel material which is based on physical interactions between charged building blocks in the form of poly(acrylic acid-co-4 acrylamidomethylbenzenesulfonic acid) and/or poly(acrylic acid-co-acrylamidoethanesulfonic acid) and/or poly(acrylic acid-co-acrylamidoethanhydrosulfate) and uncharged building blocks in the form of polymers, wherein strongly positively charged peptide sequences are conjugated to the polymers, wherein the composition of the hydrogel material can be configured on the basis of three parameters defining the building blocks carrying the charged groups selected from a group of parameters P0, P1, P2, P3. Parameter P0 corresponds to a value from the number of the ionized, anionic groups, assuming 30% ionization of all anionic groups, per unit volume of the hydrogel material swollen under physiological conditions, parameter P1 corresponds to a value from the number of the strongly anionic groups, with an intrinsic pKₛ value of less than 2.5, per unit volume of the hydrogel material swollen under physiological conditions, parameter P2 corresponds to a value from the number of the strongly anionic groups, with an intrinsic pKₛ value of less than 2.5, per repeat unit divided by the molar mass of the repeat unit, and parameter P3 corresponds to a value for describing the amphiphilia of the molecule structure surrounding the anionic groups.

In the physically crosslinked hydrogel material, it can be provided that the highly positively charged peptide sequences comprise at least ten repetitions of lysine or arginine or at least five repetitions of dipeptide motifs with lysine and alanine or arginine and alanine.

The invention also relates to a method for determining and providing a configuration for a hydrogel material for sequestering bioactive substances into the hydrogel material and depletion of the substances in a biofluid and/or release of substances from the hydrogel material into the biofluid and depletion of the substances in the hydrogel material using one of the hydrogel materials according to the invention.

In the method, substances according to a value PP, which is calculated from the ratio of the net charge of a substance and the surface of the substance accessible to water, are divided into at least two categories, and for each category, for at least two different values of a parameter of a predetermined hydrogel configuration, in each case one substance absorption value is determined experimentally on the basis of a percentage substance absorption of a test substance assigned to the category into the hydrogel material and/or a substance release value is determined experimentally on the basis of a percentage substance release of the test substance from the hydrogel material into the biofluid. On the basis of at least two substance absorption values determined experimentally and/or on the basis of at least two substance release values determined experimentally, a category-specific function is formed in each case, on the basis of which further substance absorption values and/or substance release values of further predetermined hydrogel configurations with predetermined parameters are determined, wherein, for influencing the concentration of any substance in the biofluid which can be assigned to a category, those hydrogel configurations of the hydrogel material with predetermined values are selected as suitable for the parameters for which a category-specific regression function formed from the substance absorption values determined experimentally and the determined substance absorption values and/or the substance release values determined experimentally and the calculated substance release values has a coefficient of determination R² of at least 0.6, preferably at least 0.7.

The method according to the invention makes it possible to specify a suitable hydrogel configuration according to parameters P0, P1, P2, P3 with predetermined parameter values for any desired substance, which can be assigned to a category according to its PP value, for a predetermined substance absorption value or a predetermined substance release value, without an experimental determination of the substance release value or the substance absorption value being necessary. Using the hydrogel materials according to the invention, application-specific hydrogel configurations can thus be determined and provided for the application in accordance with the determined configuration.

The calculation/determination of the PP value for a bioactive substance is calculated from the net charge of the substance divided by the substance surface accessible to the solvent water.

For protein-based substances, any protein structure is available in the Protein Data Bank (PDB, http://www.rcsb.org/). The net charge of the selected protein structure is calculated with the Delphi Web Server (http://compbio.clemson.edu/sapp/delphi webserver/) with default settings at pH 7. The protein surface accessible to the solvent water is calculated using the PyMol software (www.pymol.org) using a solvent radius of water of 1.4 Å. Then PP is calculated from the net charge divided by the protein surface accessible to the solvent water multiplied by a factor of 1000000 to obtain the unit 10⁻⁶×[1/A² or A⁻²]. For non-protein-based substances, the net charge which can be derived from the chemical structure and which corresponds to the excess of anionic or cationic groups, and the molecular surface which is accessible by the solvent water and which was derived in analogy to the formation rule for parameter P3 by using the ChemDraw 19.0 and ChemAxon MarvinSketch 19.21 software, are calculated. The value obtained is multiplied by a factor of 1000000 to obtain the unit 10⁻⁶×[1/A² or A⁻²].

According to the invention, a substance absorption value describes the percentage content of the substance content of a solution containing substances which is absorbed into a hydrogel material from a biofluid. The substance release value describes the proportion of substances bound in the hydrogel material released into a biofluid. A predetermined substance absorption value range and a predetermined substance release value range each define a value range with a minimum substance absorption value to be determined and a maximum substance absorption value to be determined, or a minimum percentage substance release value to be determined and a maximum percentage substance release value to be determined.

The invention is based on the surprising finding that substances can be categorized on the basis of a specific substance property which is calculated according to the invention by the ratio of the net charge of the substance and the surface accessible to water (PP value), wherein different hydrogel configurations can be assigned to the substances in a category which are suitable for influencing the concentration of a substance in a biofluid with respect to a predetermined percentage substance absorption value or a predetermined percentage substance release value. Since the assignment of the appropriate hydrogel configurations applies to each substance in a category, it is sufficient if suitable hydrogel configurations are determined for one respective test substance. With the method according to the invention, it is thus possible to specify at least one hydrogel configuration for a predetermined percentage substance absorption value or a percentage substance release value for any desired substance in a category without having to perform a specific experimental procedure for the substance in question.

Preferably, the predetermined hydrogel configuration can be formed with parameters P0, P2, P3 as P0P2P3 hydrogel configuration or P1, P2, P3 as P1P2P3 hydrogel configuration.

According to an embodiment variant of the method according to the invention, substances can be divided into four categories A, B, C and D on the basis of their PP value, wherein substances with a PP value >940 are assigned to category A, substances with a PP value in a range from 940 to 128 are assigned to category B, substances with a PP value in a range from 128 to −128 are assigned to category C and substances with a PP value <128 are assigned to category D.

For the experimental determination of the substance absorption values and/or for the experimental determination of the substance release values, a value in a range from 0 to 50 μmol/ml for parameter P0, a value in a range from 0 to 100 μmol/ml for parameter P1, a value in a range from 0 to 10 mmol/(g/mol) for parameter P2, and a value in a range from −7 to 7 A−2 for parameter P3 can be predetermined.

For example, the proteins eotaxine, IP10, SDF1, MCP1, SGF2, rantes, bNGF, IL-4, IL8, PGGFb, GRO-A, IL6, IL10, TNFα, IFNg, VEGF1 65 MP, IL1b, GMCSF, IGF can be used as test substances.

According to the method according to the invention, it can be provided that a value range for at least one parameter P0, P1, P2 or P3 of a hydrogel configuration is determined on the basis of the category-specific regression function for substance absorption values and/or substance release values which are not determined experimentally, i.e. for assigned substance absorption values and/or substance release values. The values of the parameters are determined using mathematical models.

Further, it can be provided that the values of the parameters for the hydrogel configuration P0P1P2 and/or for the hydrogel configuration P1P2P3 are selected in such a way that in the resulting hydrogel at least one substance of a category is bound only up to 50% of an initial concentration in the hydrogel material or released therefrom.

The hydrogel materials according to the invention are intended for use for factor management in vivo for controlling angiogenesis, in immune diseases, cancers, diabetes, neurodegenerative diseases, Crohn's disease, ulcerative colitis, multiple sclerosis, asthma, rheumatoid arthritis or cutaneous wound healing and bone regeneration.

Furthermore, the hydrogel materials according to the invention can be used for the targeted purification of proteins from cell lysates of microbial or eukaryotic origin.

Use of the hydrogel material according to the invention for the in vitro cell and organ culture of induced pluripotent stem cells (IPS cells), as well as further stem cells and precursor cells not to be assigned iPS, primary cells obtained from patients, immortalized cell lines, as well as heart tissue, muscle tissue, kidney tissue, liver tissue and nerve tissue in which the respective cells/organs are polymerized into the hydrogel or cultivated on the surface of the hydrogel.

Cells can be immobilized into the hydrogel material according to the invention.

Further details, features and advantages of designs of the invention will become apparent from the following description of example embodiments with reference to the associated drawings and tables. The following is shown:

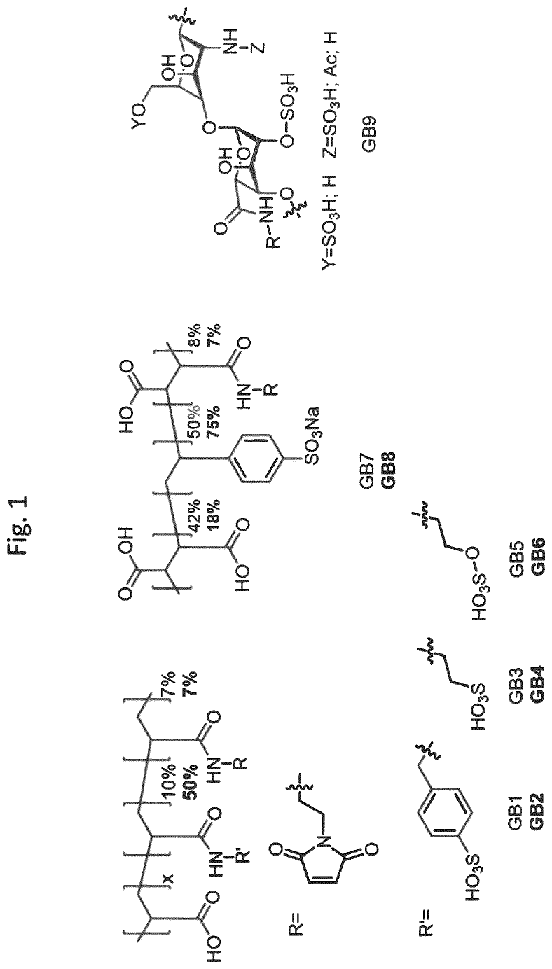

FIG. 1: Chemical structures of the anionic building blocks used for hydrogel formation, left: derivatives of polyacrylate (PAA) with different radicals: R: maleimide group and R′=aminomethyl-benzene-1-sulfonic acid (AMBS, GB1 and GB2) or aminoethanesulfonic acid (AES, GB3 and GB4) or 2 aminoethyl hydrogen sulfate (AHS, GB5 and GB6), center: styrene sulfonate-maleic acid copolymers with radical R: maleimide group, (GB7 and GB8), right: heparin (GB9),

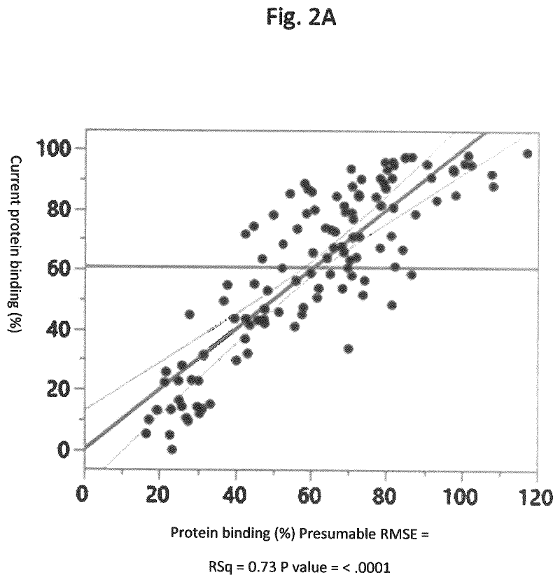

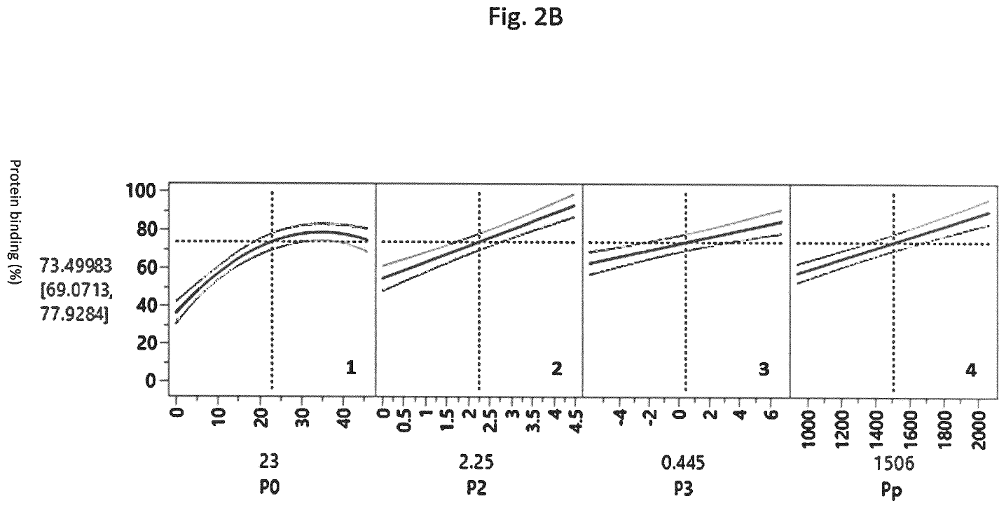

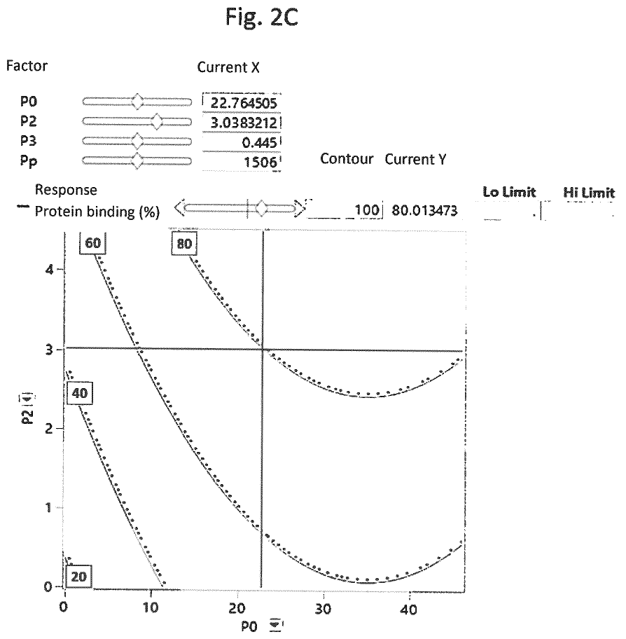

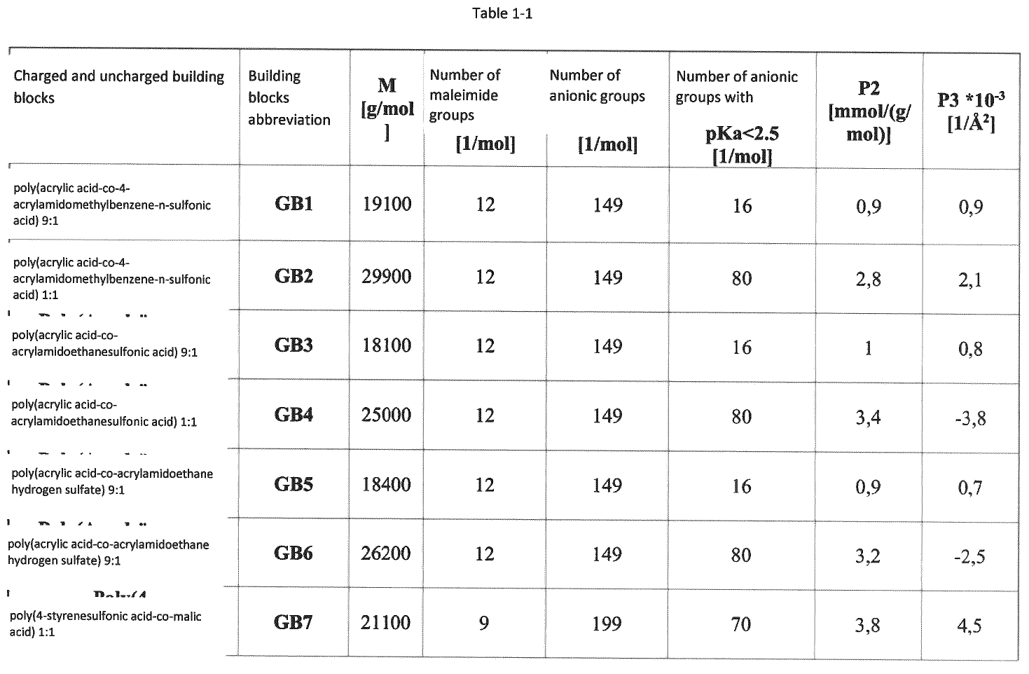

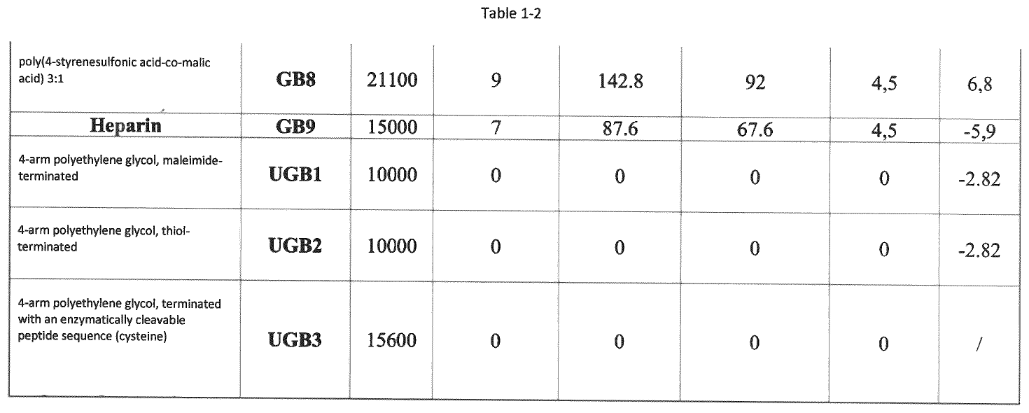

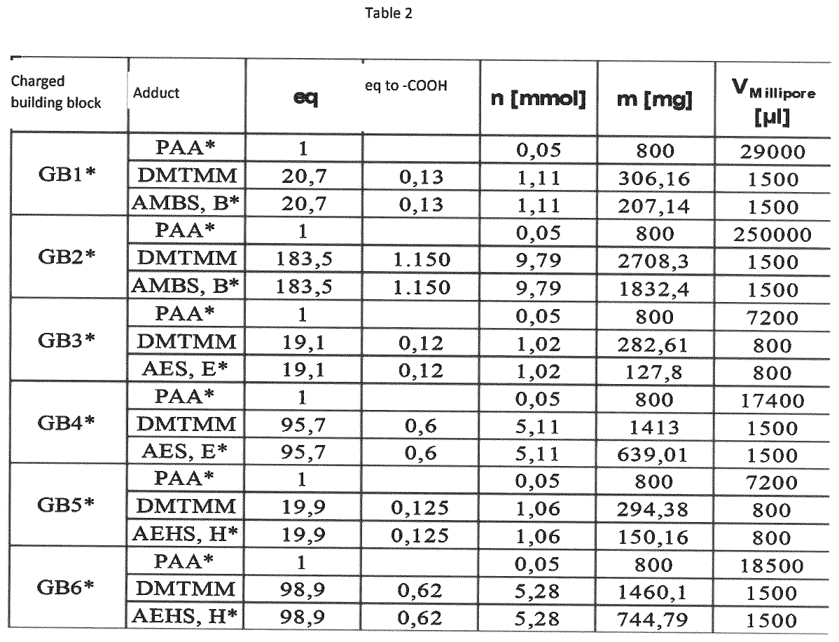

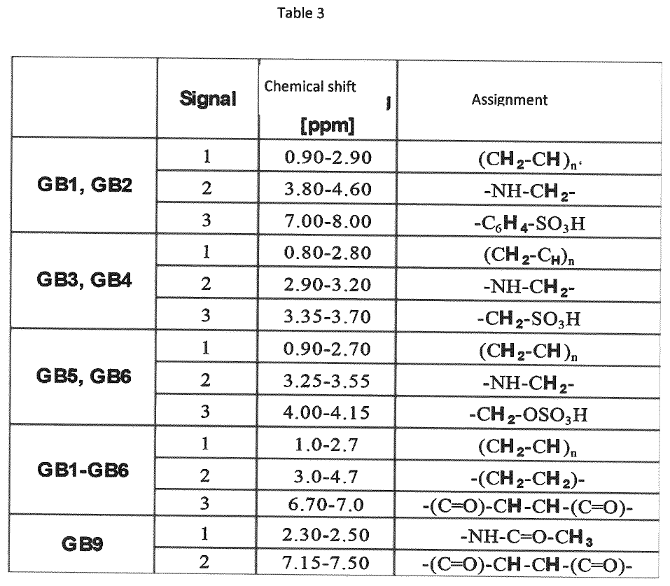

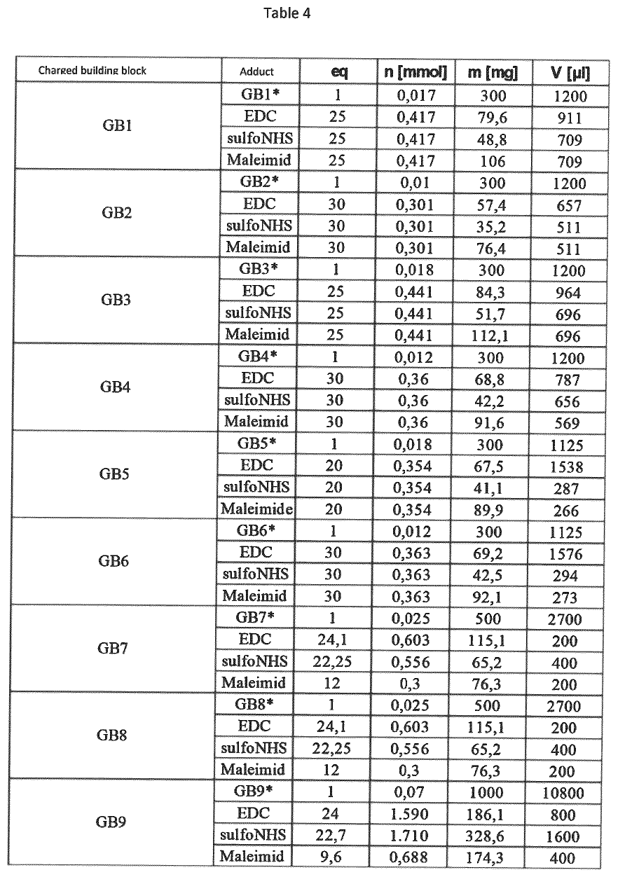

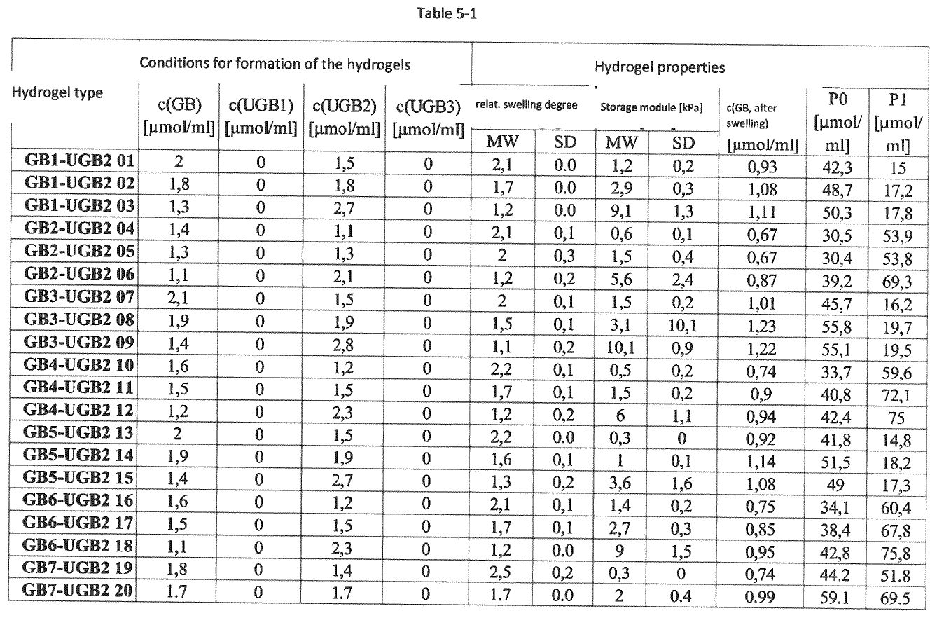

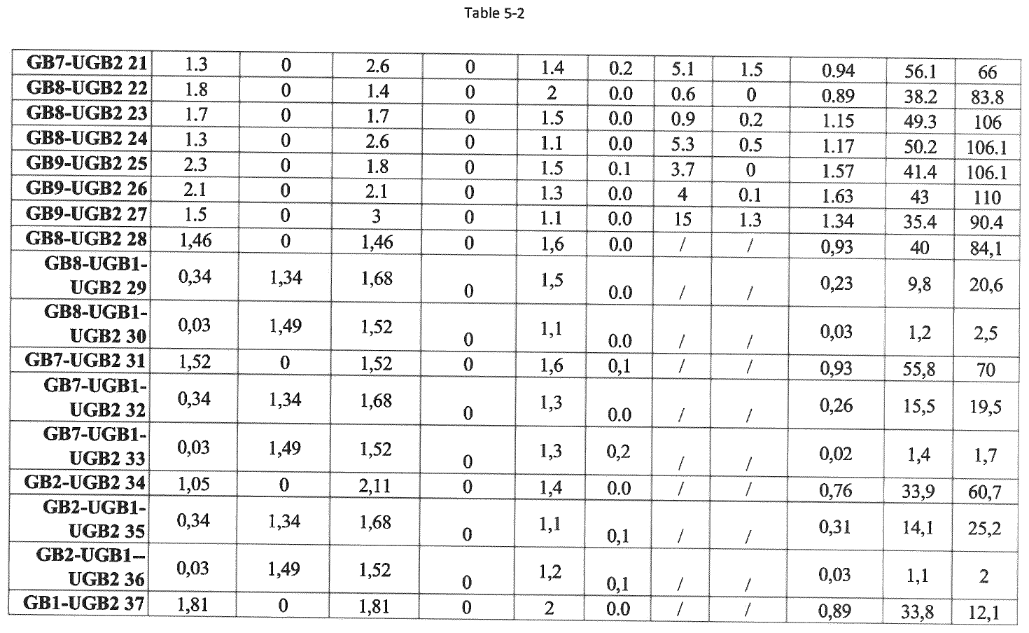

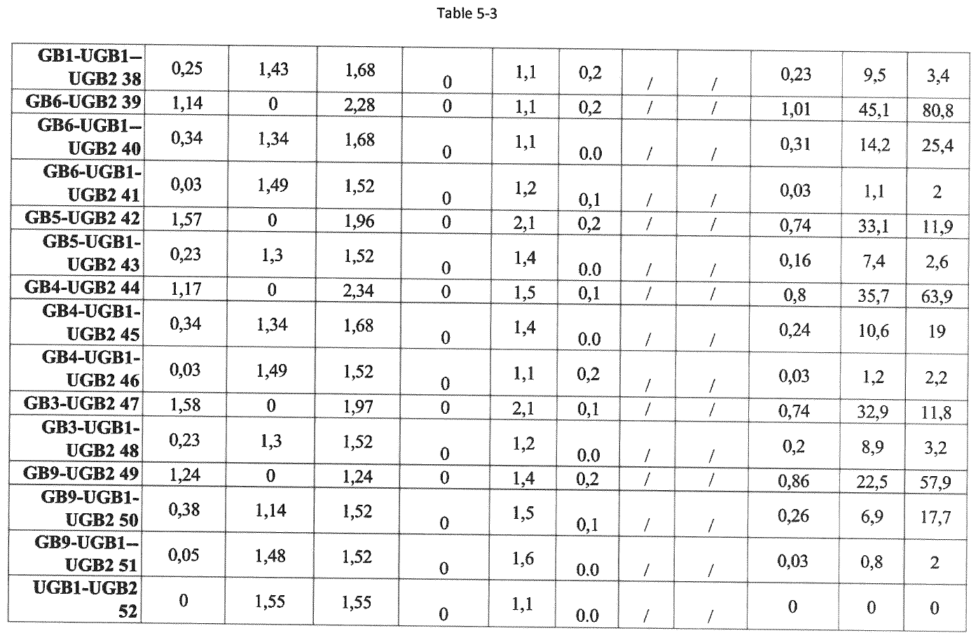

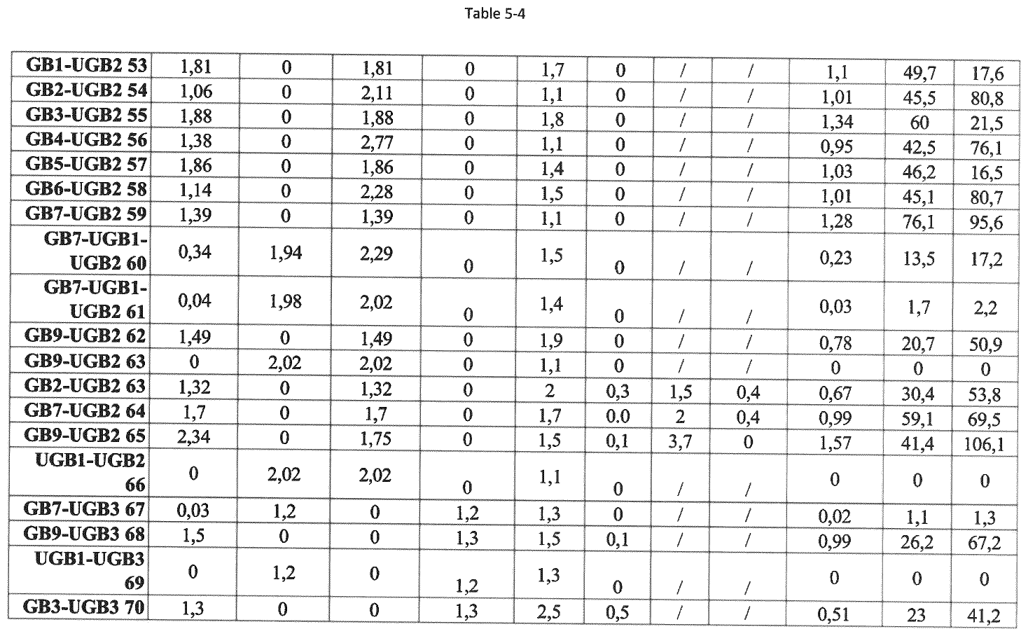

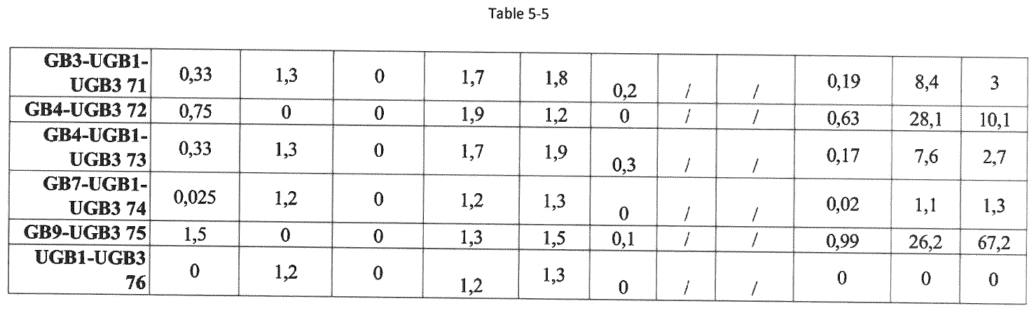

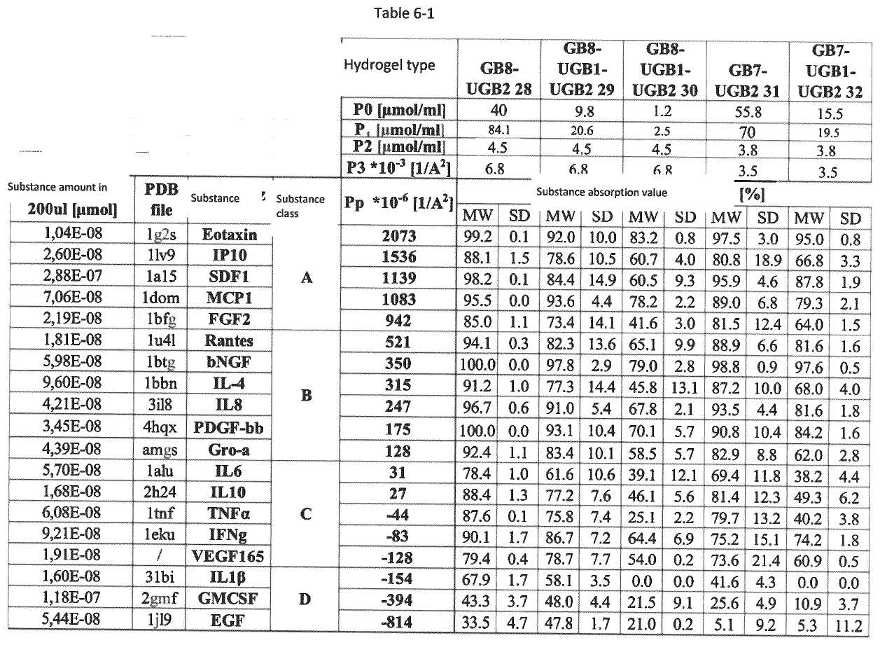

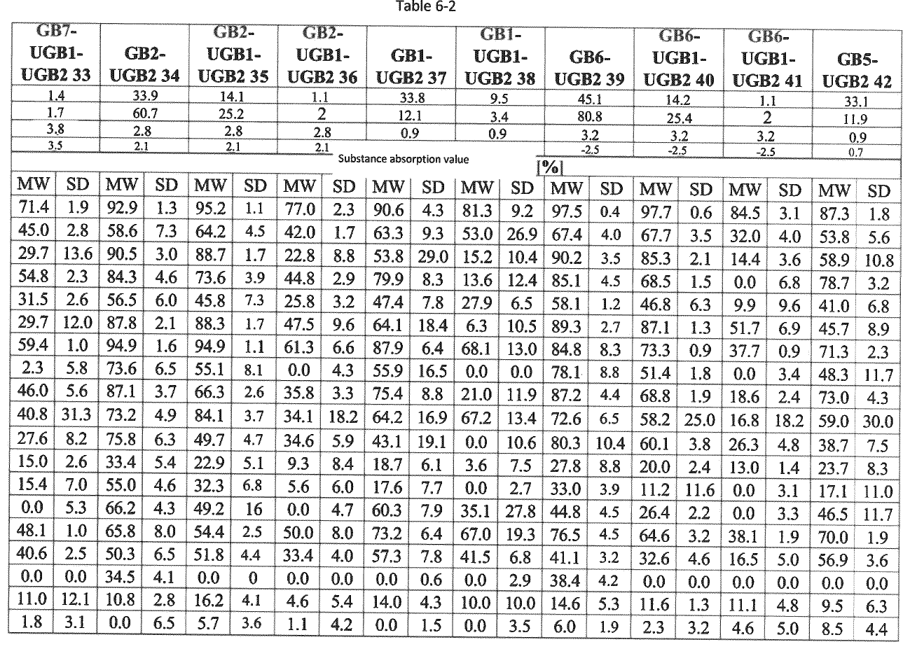

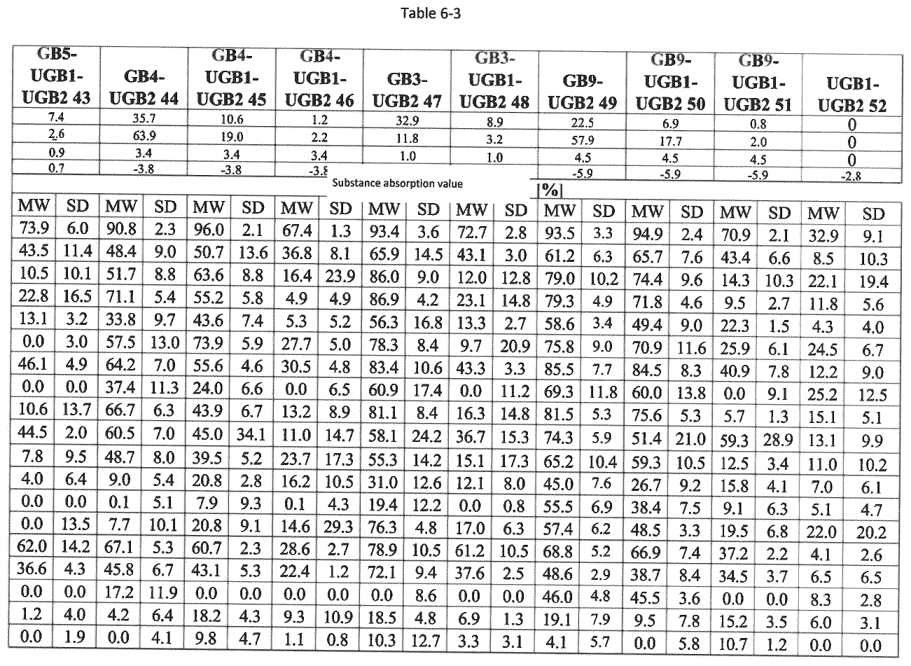

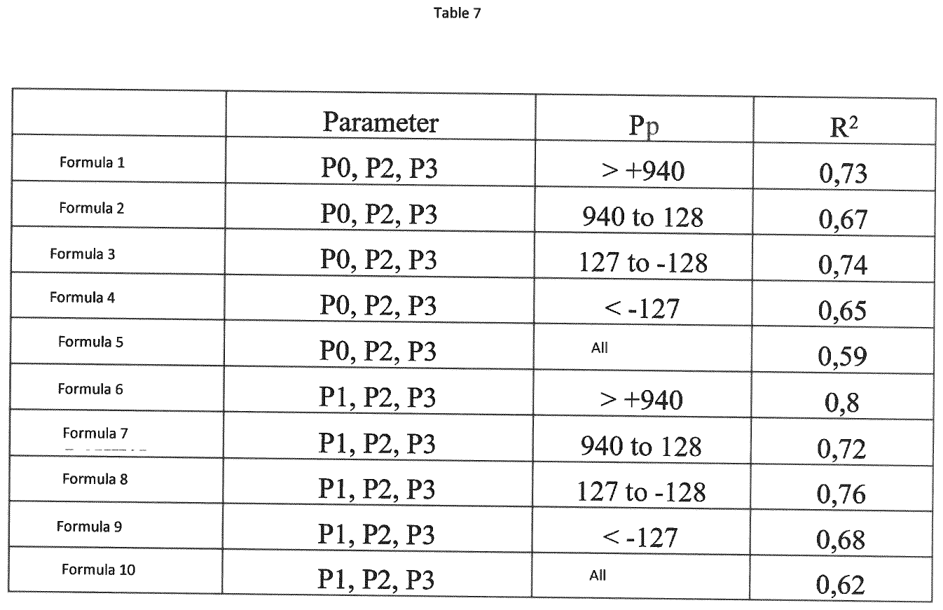

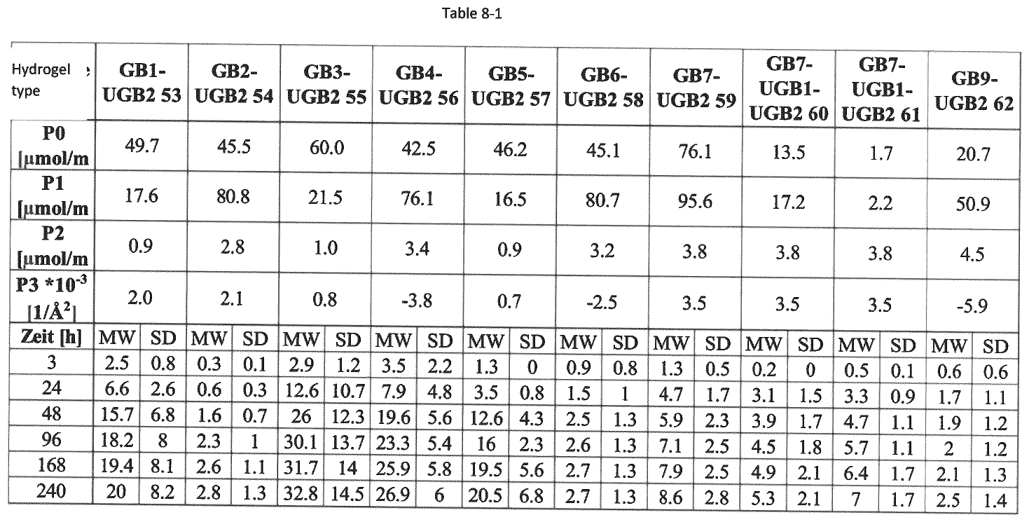

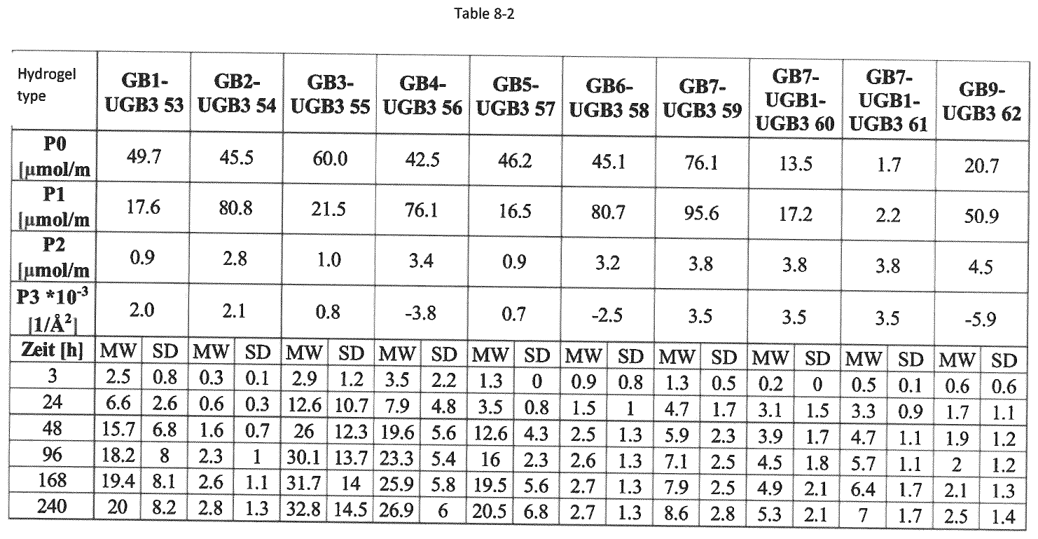

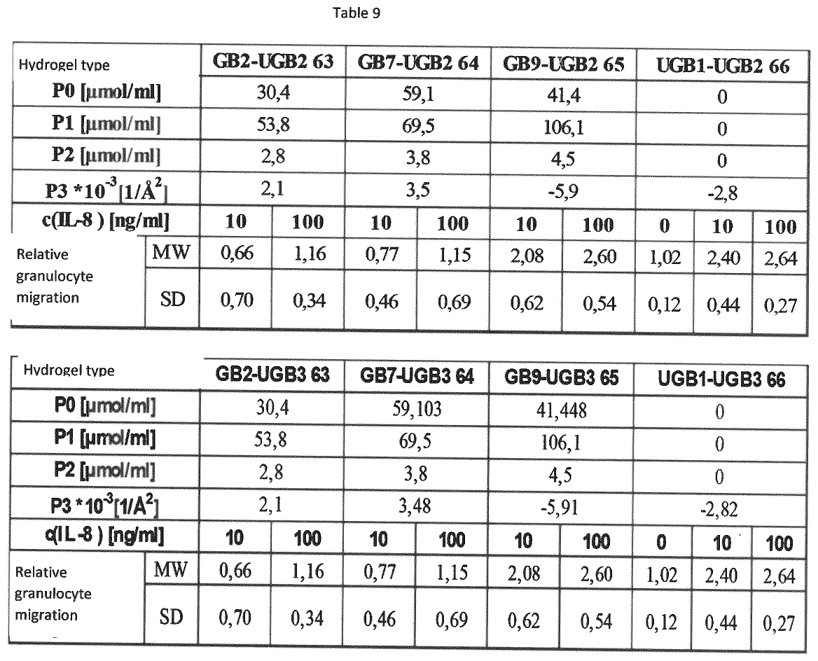

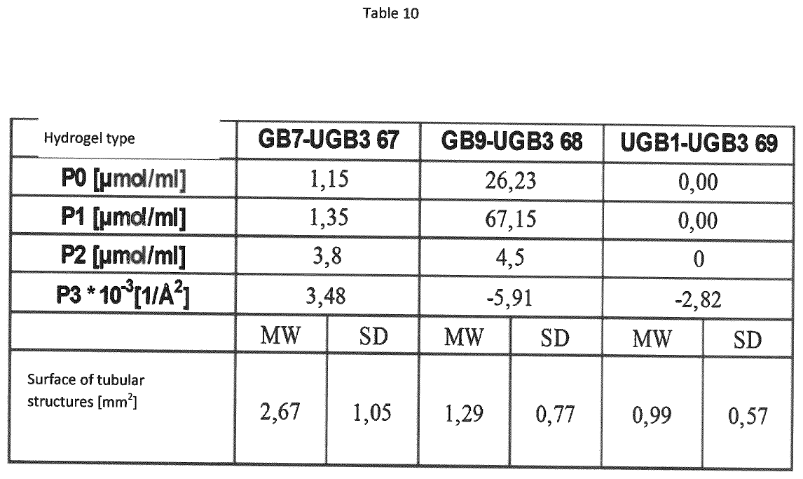

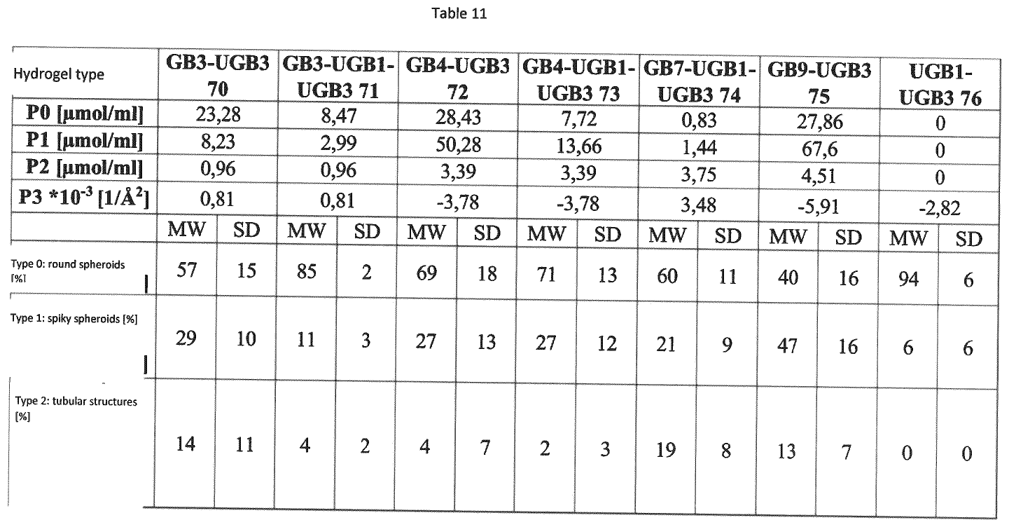

FIGS. 2A-C: representations to explain the method according to the invention,

Tables 1-1 to 1-2: hydrogel building blocks used: columns of the table from left to right: (1) chemical name, (2) abbreviation, (3) molar mass, (4) number of maleimide groups per molecule, (5) number of anionic groups per molecule, (6) number of strongly anionic groups with pKa<2.5 per molecule, (7) parameters P2 and (8) P3 of the hydrogel building blocks,

Table 2: batch sizes for the sulf(on)ation of the polysodium acrylate with sulf(on)ated amines by means of DMTMM, column (1) name of the anionically charged building block, (2) name of the adducts: the equivalents (eq) used, based on the amount of each polymer and the carboxylic acid groups, the amount of substance, the volume of millipore water as solvent and the solids content,

Table 3: signals in the 1H NMR spectrum (500.13 MHz in D2O) for the anionic charged building blocks GB1 and GB2, GB3 and GB4, GB5 and GB6 as well as GB9 with the corresponding chemical displacement and assignment of the based protons,

Table 4: synthesis conditions for the maleimidation of the anionically charged building blocks GB1 to GB9, GB* denote the respective non-maleimized anionically charged building blocks,

Tables 5-1 to 5-5: a rule for the formation of hydrogel materials of types 1 to 76 and resulting physicochemical properties of hydrogel materials swollen under physiological conditions (column 1): name of the hydrogel material type: 1 to 76, (columns 2-4): concentration of the hydrogel building blocks (GB1, 2, 3, 4, 5, 6, 7, 8 or 9, UGB1, UGB2, UGB3) for polymer network formation, (columns 5 and 6): relative degree of swelling and standard deviation for swelling of hydrogel materials under physiological conditions (columns 7 and 8): storage module and standard deviation of hydrogel materials swollen under physiological conditions (column 10): concentration of the anionically charged building blocks after swelling under physiological conditions (column 11): parameter P0 hydrogel materials swollen under physiological conditions (column 12): parameter P1 of the hydrogel materials swollen under physiological conditions,

Tables 6-1 to 6-3: the sequestration of bioactive substances: top 4 rows: (1) quantity of substances of the bioactive substances used (here proteins) in 200 μl solution which has been brought into contact with the respective hydrogel material, (2) file of the protein data bank (PDB), (3) name of the bioactive substance, (4) category A to D, (5) parameter PP of the bioactive substances, (6 to 25, resulting sequestration of the bioactive substances by the hydrogel materials, lower 18 rows: description of the anionic hydrogel materials by their names (hydrogel material types 28-52) and parameters P0, P1, P2 and P3), and of the substance absorption value (in %, mean±standard deviation, n=6,

Table 7: linear regression analysis of the sequestration values, (1) number of the formula, (2) hydrogel parameters taken into account, (3) PP values (substances) taken into account, (4) coefficient of determination R² of the function with respect to the substance release values

Tables 8-1 to 8-2: cumulative release of VEGF165 as a bioactive substance from hydrogel materials of types 53 to 62. (mean±standard deviation, n=6), top row: hydrogel material types, rows 2 to 5: parameters P0, P1, P2, P3, rows 7 to 12: substance release values in % at 3, 24, 48, 96, 168 and 240 h,

Table 9: migration of immune cells from human whole blood and reaction of blood granulocytes to IL-8 solution incubated with various anionically charged building blocks, relative to the number of cells without IL-8 which migrated into UGB1/UGB3 negative control hydrogel materials. (mean±standard deviation, n=5),

Table 10: cell culture of HUVECs, row 1: hydrogel type, rows 2-4: parameters P0 to P3, row 6: surface of tubular structures as desired cell morphology (mean±standard deviation, n=5), and

Table 11: cell culture of HK-2 cells, row 1: hydrogel type, rows 2-4: parameters P0 to P3, row 6: round spheroids in %, row 7: spiky spheroids in %, row 8: tubular structures in % (mean±standard deviation, n=5).

Anionically charged hydrogel building blocks are abbreviated as GB in the following, wherein different anionically charged building blocks are additionally identified by a reference numeral. Uncharged building blocks of the hydrogel material are abbreviated as UGB, wherein different uncharged building blocks are identified by a reference numeral. For the sake of simplicity and for brevity, hydrogel materials are referred to in the tables as hydrogels. Hydrogel material types are referred to as hydrogel types in the tables.

FIG. 1 shows chemical structures of the anionic building blocks used for hydrogel material formation. On the left, derivatives of polyacrylate (PAA) with various radicals: R: maleimide group and R′=aminomethyl-benzene-1-sulfonic acid (AMBS, GB1 and GB2) or aminoethanesulfonic acid (AES, GB3 and GB4) or 2 aminoethyl hydrogen sulfate (AHS, GB5 and GB6) are represented. In the center, styrene sulfonate-maleic acid copolymers with radical R: maleimide group, (GB7 and GB8) are represented. Heparin (GB9) is represented on the right. The anionic building blocks used to prepare the hydrogel materials are sulfated or sulfonated polymers based on polyacrylate (PAA, see FIG. 1), GB1 to GB6, styrene sulfonate-maleic acid copolymers GB7 and GB8 or heparin (GB9).

Tables 1-1 to 1-2 show examples of hydrogel building blocks for the preparation of hydrogel materials according to the invention. Columns 1 to 8 of Tables 1-1 to 1-2 show from left to right: Column 1 is the chemical name of the hydrogel building blocks, column 2 is the abbreviation applicable herein, column 3 is the molar mass, column 4 is the number of maleimide groups per molecule, column 5 is the number of anionic groups per molecule, column 6 is the number of strongly anionic groups with pKa<2.5 per molecule, column 7 is parameter P2 and column 8 is parameter P3 of the hydrogel building blocks.

### Synthesis of the Anionically Charged Building Blocks GB1 to GB6

The anionically charged hydrogel building blocks based on PAA (GB1 to GB6) are synthesized by amide formation mediated by 4-(4,6-dimethoxy-1,3,5-triazin-2-yl)-4-methylmorpholinium chloride (DMTMM) according to the protocol of (Thompson, K. et al. J. Polym. Sci. Part A Polym. Chem. 44, 126-136 (2006). Three different sulf(on)ated amines, 4-aminomethyl-benzene-1-sulfonic acid (AMBS, for the formation of GB1 and GB2), aminoethanesulfonic acid (AES, for the formation of GB3 and GB4) and 2 aminoethyl hydrogen sulfate (AEH, for the formation of GB5 and GB6) are each conjugated to PAA* at 10% or 50%, based on the number of carboxyl groups, in order to vary the local charge distribution of the charged groups along the polymer backbone. The amines of GB3-GB6 have very similar molecular structures with the only difference between the terminal sulfonate group in GB3 and GB4 and a terminal sulfate group in GB5 and GB6. The sulfonate group in 4-aminomethyl-benzene-1-sulfonic acid is bonded to a phenyl group, which increases the hydrophobic character of the anionic building blocks GB1 and GB2 (see parameter P3, Tables 1-1 to 1-2).

For synthesis, polysodium acrylate (15 kDa, 34.7% by weight in H2O, Sigma-Aldrich, Germany) was freeze-dried, 800 mg were presented for functionalization and dissolved in millipore water (Table 2). Subsequently, the DMTMM (TCI Chemicals, Japan) which has been thawed in the desiccator is first added dry, followed by the amine, 4-aminomethyl-benzen-1-sulfonic acid (AMBS, Sigma-Aldrich), aminoethanesulfonic acid (AES, Sigma-Aldrich, Germany) or 2 aminoethyl hydrogen sulfate (AEH, Sigma-Aldrich, Germany) and the vessel with the stated volume is rinsed in each case. The reaction mixture is stirred at room temperature (RT) overnight. The precipitate, which is in part precipitated, is dissolved with millipore water and the solution is dialyzed in a dialysis membrane with an exclusion size (MWCO) of 2 kDa (ZelluTrans, Roth, Germany) for 6 h against 1 M NaCl solution (NaCl, Fluka, Germany) with a double change of medium, then for 18 h against millipore water with a three-fold change and then freeze-dried. To determine the degree of functionalization, the purified product is analyzed by means of 1H NMR in D2O (DRX 500 from Bruker at 500.13 MHz, signal of the solvent serves as reference for the chemical shift), wherein the ethyl protons for the polymers E and H, the ring protons for B are considered as an internal reference (see Table 3). The degree of functionalization can be calculated with the ratio to the three protons of the polymer backbone.

Table 2 shows batch sizes for the sulf(on)ation of the polysodium acrylate with sulf(on)ated amines by means of DMTMM, column 1 name of the anionically charged building block, column 2 the name of the adducts: with regard to the equivalents (eq) used, based on the amount of substance of the respective polymer and the carboxylic acid groups, the amount of substance, the volume of millipore water as solvent and the solids content.

In order to analyze the conversion of the sulf(on)ation reaction, a proton nuclear spin resonance spectroscopy analysis (1H-NMR) of each polymer was carried out (see Table 3). The 1H-NMR spectra of the sulf(on)ated conjugates showed characteristic signals between 2.9 ppm and 3.70 ppm for GB3*/GB4* and signals between 3.25 ppm and 4.15 ppm for GB5*/GB6* corresponding to the four protons of the ethyl linker, while for GB1*/GB2* signals of 7.00-8.00 ppm, based on the phenyl protons, were used for the calculation. The integration of the characteristic signals in relation to the signal area of the three protons of the PAA backbone of 0.80-2.90 ppm made it possible to determine the degree of functionalization of the PAA with various sulf(on)ated amines.

Table 3 shows signals in the 1H NMR spectrum (500.13 MHz in D2O) for the anionic charged building blocks GB1 and GB2, GB3 and GB4, GB5 and GB6 as well as GB9 with the corresponding chemical displacement and assignment of the based protons.

### Maleimization of the Anionically Charged Building Blocks GB1 to GB9

In order to enable the formation of a network in the example with thiol-functionalized 4-arm polyethylene glycol (UGB2, starPEG-SH), the respective anionically charged building block (GB1 to GB9) with N-2-aminoethylmaleimide trifluoroacetic acid salt (maleimide) is coupled to the carboxyl groups activated with 1-ethyl-3-(dimethylaminopropyl)- via 1-ethyl-3-(dimethylaminopropyl)-carbodiimide/N-hydroxysulfosuccinimide (EDC/sulfoNHS), resulting in approximately 12.5 maleimide groups in GB1 to GB6, 9 per GB7 and GB8 and 7 per GB9 (see Tables 1-1 to 1-2, column: number of maleimide groups).

For this purpose, 300 mg of polymer are dissolved in the corresponding volume of solvent, then sulfo-NHS (Sigma-Aldrich, Germany) and afterwards EDC (Iris Biotech, Germany) are then added in solution and stirred for 20 min (10 min for GB7) in an ice bath before dissolved N-(2-aminoethyl)maleimide trifluoroacetate (Maleimide, Sigma-Aldrich, Germany) is slowly added dropwise. Ice-cold millipore water is used as solvent in each case. The reaction mixture is stirred in an ice bath for a further 10 minutes, and further overnight at room temperature. The solution is dialysed in a 2 kDa MWCO dialysis membrane for 6 h against 1 M NaCl solution with a double change of medium, then for 18 h against millipore water with a three-fold change and then freeze-dried. Further information on the employed reaction mixtures is shown in Table 4. The determination of the number of the maleimide groups is via 1H-NMR in D2O, wherein for the sulf(on)ated polymers, the number of the protons of the polymer backbone, for heparin, the number of the acetyl protons are set in relation to the maleimide protons (see Table 3). The degrees of functionalization are determined in each case via 1H NMR (see Table 3). Only for GB7 and GB8, the degree of maleimide conjugation is evaluated by the peptide titration method. (see Atallah et al.,2018 doi:10.1016/j.biomaterials.2018.07.056), The conjugates are reacted with various molar equivalents of cysteine-containing peptides and analyzed by size exclusion chromatography.

Table 4 shows synthesis conditions for maleimizing the anionically charged building blocks GB1 to GB9, wherein GB* denote the respective non-maleimized anionically charged building blocks.

### Formation and Crosslinking of Hydrogel Materials of Types 1 to 76

In order to form the hydrogel materials according to the invention, the respective maleimide-functionalized, anionically charged building block GB1, GB2, GB3, GB4, GB5, GB6, GB7, GB8 or GB9 or a mixture of GB1, GB2, GB3, GB4, GB5, GB6, GB7, GB8 or GB9 with UGB1 either with the thiol-functionalized UGB2 (4-arm polyethylene glycol, thiol-terminated) or with the thiol-functionalized UGB3 (4-arm polyethylene glycol, terminated with an enzymatically cleavable peptide sequence which contains cysteine) is crosslinked via a Michael type addition through mixing and reaction of the thiol groups with the maleimide groups. The hydrogel material of type 52 is formed from UGB1 and UGB2 as an uncharged reference hydrogel. The concentration of the hydrogel building blocks which are used to form the respective hydrogel material from the aqueous solutions of the hydrogel building blocks is shown in Tables 5-1 to 5-5. The reaction and covalent network formation take place within a few seconds up to one minute. The formation rules were chosen such that after swelling under physiological conditions (0.154 mmol NaCl, buffered with phosphate buffer to pH 7.4), a whole bandwidth of different parameter values for parameters P0 and P1 result, while at the same time values of different variables for parameters P2 and P3 are also defined via the respective anionically charged building block (see Tables 5-1 to 5-5, hydrogel types 1-76 and Tables 1-1 to 1-2 with respect for the values for parameters P2 and P3). In addition, the physical properties of the swollen hydrogel materials, the relative degree of swelling of the hydrogel materials and the storage module of the hydrogel materials are investigated by means of rheometry. From the relative degree of swelling and the concentration of the anionically charged building blocks during hydrogel formation, the concentration of the anionically charged building blocks after swelling is determined and parameters P0 and P1 of the hydrogel materials are calculated from this in accordance with the definition of the parameters. The physicochemical properties of the hydrogel materials for stiffness, swelling and the values of parameters P0, P1, P2 and P3 can be graded over a wide range and thus for the desired sequestration properties or absorption properties and/or release properties of bioactive substances (see Tables 5-1 to 5-5 and Tables 1-1 to 1-2).

From the indicated storage modules of the hydrogel materials, according to the rubber elasticity model (Rubinstein et al., 2003 ISBN: 9780198520597), mesh widths between 6,5 and 23 nm according to the following equation are obtained:

$$ξ = {( \frac{{G}^{′} {N}_{A}}{R T} )}^{- 1 / 3} ,$$

wherein G′ is the storage module in Pa, NA is the Avogadro constant, R is the universal gas constant and T is the temperature in Kelvin. The bioactive substances discussed here with a molecular size of less than 70 kDa should not be subject to any sterically restrictions with respect to penetration of the hydrogel material according to the invention with these mesh sizes.

For the formation of the hydrogel material, the hydrogel building blocks, charged and uncharged building blocks, are dissolved in phosphate-buffered saline (0.154 mmol NaCl, buffered with phosphate buffer to pH 7.4) in the concentration given in Tables 5-1 to 5-5 and are then mixed intensively by pipetting. The hydrogel materials form within a few seconds up to one minute.

For the determination of the physical hydrogel properties, round 9 mm cover glasses are hydrophobically coated. For coating, the cover glasses are immersed in Sigmacote® solution (Sigma-Aldrich, Germany) for 2 s, dried on filter paper and rinsed with millipore water and dried again. The solution with the anionically charged building blocks and the uncharged building blocks are dissolved in phosphate-buffered saline at pH 7.4 (PBS) and 33.5 μl of the GB solution and the UGB solution are mixed in each case, pipetted onto a cover glass and covered with a further cover glass in order to form a gel about 1 mm high. After a period of 1 h, the cover glasses are removed and swollen overnight in phosphate-buffered salt solution (PBS in the following). Rheological investigations for determining the memory module (Ares LN2, TA Instruments, United Kingdom) were carried out with swollen hydrogels punched to 8 mm with a parallel plate-plate measuring arrangement at RT, wherein the frequency was increased from 1 to 100 rad/s with a slight deformation (2%). A triple determination is performed and the mean of the storage module is formed over the entire frequency range. The loss module is smaller by several orders of magnitude (data not shown). For the determination of the relative volume swelling, the swollen gel disks are imaged with the help of a laser scanner (Fujifilm FLA-5100) and the diameter is determined. Using the following equation:

$$Q = {( \frac{d}{{d}_{0}} )}^{3} ,$$

the relative degree of swelling (see Table 3) is determined, wherein d0 corresponds to the initial diameter of the unswollen gel disk and d corresponds to the diameter after swelling in PBS for 24 h.

Tables 5-1 to 5-5 show a specification for the formation of the hydrogel materials of the types 1 to 76 and resulting physicochemical properties of the hydrogel materials swollen under physiological conditions, column 1: Name of the hydrogel material type: 1 to 76, columns 2-4: concentration of the hydrogel building blocks in the reaction mixture (GB1, 2, 3, 4, 5, 6, 7, 8 or 9, UGB1, UGB2, UGB3) for polymer network formation, columns 5 and 6: relative degree of swelling and standard deviation for swelling of hydrogel materials under physiological conditions, columns 7 and 8: Storage module and standard deviation of the hydrogel materials swollen under physiological conditions, column 10: Concentration of the anionically charged building block after swelling under physiological conditions, column 11: Parameter P0 of the hydrogel materials swollen under physiological conditions, column 12: Parameter P1 of the hydrogel materials swollen under physiological conditions.

### Characterization of the Sequestration of Bioactive Substances in the Anionically Charged Hydrogels of Types 28 to 52 by Means of Multiplex Immunoassay

A defined mixture of bioactive substances, i.e. a mixture of biomedically important protein-based signal molecules with different charge properties, is used in the corresponding binding studies with a number of different anionically charged hydrogel materials of types 28-51 and the uncharged reference gel of type 52 (see Tables 5-1 to 5-5). The recombinant proteins (reconstituted with several ProcartaPlex simplex protein standards, cat. No: EPX01A-xxxx- 901 Thermo Fisher Scientific, USA) are dissolved in PBS with 1% BSA and 0.1% proclin (Sigma-Aldrich, Germany) in various biologically relevant concentrations in the range from 0.44-12.25 ng/ml. 10 μl of each gel type (n=3) are incubated with 250 μl of the protein mixture for 24 hours, and the cytokine concentration in the supernatant is then analyzed with ProcartaPlex Simplex multiplex kits (ThermoFischer, USA) using a Bioplex 200 (Biorad, USA). Solutions without hydrogel materials are used as the control experiment and the detected amounts of protein are compared with those in solutions with hydrogel materials after incubation in order to determine the percentage of protein which has been sequestered by the hydrogel materials, i.e. absorbed. The results of the sequestration investigations are represented in Tables 6-1 to 6-3.

Tables 6-1 to 6-3 show the sequestration of bioactive substances: top 4 rows: (1) Quantity of substances of the bioactive substances used (here proteins) in 200 μl solution which has been brought into contact with the respective hydrogel material, (2) file of the Protein Data Bank (PDB), (3) name of the bioactive substance, (4) category A to D, (5) parameter PP of the bioactive substances, (6 to 25) resulting sequestration of the bioactive substances by the hydrogel materials, Lower 18 rows: Description of the anionic hydrogel materials based on their names (hydrogel material types 28-52) and parameters P0, P1, P2 and P3), and of the substance absorption value (in %, mean value±standard deviation, n=6).

From the results of Tables 6-1 to 6-3, the substances are divided into four categories A, B, C and D as a function of their PP value, wherein substances with a PP value greater than 940 are assigned to category A, substances with a PP value in a range from 940 to 128 are assigned to category B, substances with a PP value in a range from 128 to −128 are assigned to category C and substances with a PP value less than −128 are assigned to category C. Subsequently, a linear regression analysis is carried out by means of the statistical analysis software JMP (SAS Institute) according to the method of the smallest squares between the respective percentage value of the substance absorption (as y-value) and parameters P0 or P1 and P2 and P3 of the hydrogels and the values for PP of the substances.

The aim of linear regression analysis is to describe the influence of P0 or P1 and P2 and P3 and PP on the substance absorption in a mathematical function as set out in the following equation 1:

$$y = α + {C}_{0} {P}_{0} + {C}_{2} {P}_{2} + {C}_{3} {P}_{3} + {C}_{p} {P}_{p} + {C}_{00} {P}_{0}^{2} + {C}_{22} {P}_{2}^{2} + {C}_{33} {P}_{3}^{2} + {C}_{pp} {P}_{p}^{2} + {C}_{02} {P}_{0} {P}_{2} + {C}_{03} {P}_{0} {P}_{3} + {C}_{0 p} {P}_{0} {P}_{p} + {C}_{23} {P}_{2} {P}_{3} + {C}_{2 p} {P}_{2} {P}_{p} + {C}_{3 p} {P}_{3} {P}_{p} \qquad Equation 1$$

$$y = substance absorption$$

$$α = axis section$$

$$C = coefficients$$

The results of the experimental sequestration experiments (e.g. for all substances or only for substances in a category A or B or C or D) are introduced into the regression analysis as pairs of values consisting of the respective substance absorption value in % and the associated parameter values for P0 or P1, P2, P3 and PP (see Tables 6-1 to 6-3), and a mathematical relationship (function) is formed as represented above in equation 1. The numerical values (as constants) of the axis section and the coefficients of the parameter terms are determined by iteratively comparing the substance absorption values determined on the basis of the functional equation with the experimental results read in the pairs of values (Tables 6-1 to 6-3). The differences between predicted and measured values are called residuals. By using the method of the smallest squares, the values of the axis section and the coefficients are determined by iteratively comparing the substance absorption values determined on the basis of the functional equation with the experimentally determined substance absorption values and squaring the distance between experimental substance absorption values and ones calculated by the function and minimizing the sum of these squared distances.

FIG. 2A shows how the least squares approach is displayed and evaluated in the JMP software. The measured values (y-axis) are plotted against the values (x-axis) predicted by the function. Each point represents one of the experiments performed on which the model is based, and ideally all points should be on the red line (y=x). The farther each point is from the red line, the higher the residue for this particular experimental point. Rsq (R²) is used to evaluate the quality of the model. The more accurate the model, the lower the residuals and the higher the coefficient of determination; Rsq value (between 0 and 1).

A total of 10 regression analyzes were carried out which, according to the selected parameters and substances which can be taken from Table 7, have the coefficients of determination R² set out there.

The functions have the corresponding parameters P0 or P1 and P2 and P3 as well as PP in their weighting. For each function, both the linear contributions of the individual parameters and the square effects between the parameters are taken into account. The formulas presented show only the significant contributions (p<0.01).

Equation 2 shows, by way of example, the significant contributions of P0, P2 and P3 to the absorption of substances in category A. The function determined thereby describes the relationship between substance absorption and the parameters and can therefore also be used for predicting new substance absorption values as a function of predetermined parameter values P0 or P1, P2, P3 and PP. When a specific bioactive substance is predetermined and corresponding to a discrete value PP, it is also possible to derive different parameter combinations P0 or P1 and P2 and P3 for the configuration of a specific anionically charged hydrogel material for any desired substance sequestration value or substance absorption value. Overall, therefore, any desired sequestering properties can be predicted and new combinations of properties of materials can be developed and predicted.

$$P 0 , P 2 and P 3 of substances in category A : \qquad Equation 2$$

$$Substance absorption [ \% ] = 73.500 + 19.179 \cdot ( \frac{P 0 - 23}{23} ) + 19.157 \cdot ( \frac{P 2 - 2.25}{2.25} ) + 10.960 \cdot ( \frac{P 3 - 0.445}{5.355} ) + 16.122 \cdot ( \frac{Pp - 1506}{566} ) + ( \frac{P 0 - 23}{23} ) \cdot ( \frac{P 0 - 23}{23} ) \cdot ( - 18.349 ) + ( \frac{P 0 - 23}{23} ) + ( \frac{Pp - 1506}{566} ) \cdot ( - 9.097 )$$

These predictions are possible as follows using the JMP® software: For the equation (represented here in the example of equation 2), four cases are represented in which only one parameter on the X-axis is considered as a variable and constant values are set for the other parameters (see FIG. 2B). Thus, depending on one parameter, all substance absorption values from the minimum to the maximum can be determined when the other three parameters are given. See, for example, FIG. 2B on the left for the value P0.

Furthermore, the dependence of 2 variable parameters on one another can also be examined. This is then correspondingly a two-dimensional representation of the substance absorption values as a function of both parameters, as is represented in FIG. 2C. Lines of constant substance absorption values (so-called isolines) are obtained, wherein one surface contains two constant parameters. With three parameters, a cube (a respective variable parameter along the cube axis and a constant parameter) would result which is created from stacked surfaces of 2 variable parameters. For a substance with discrete PP, a cube with the variable parameters P0 or P1, and P2 and P3 would accordingly result.

$$P 0 , P 2 and P 3 of substances in category A : \qquad Equation 1$$

$$Substance absorption [ \% ] = 73.5 + 19.179 \cdot ( \frac{P 0 - 23}{23} ) + 19.157 \cdot ( \frac{P 2 - 2.25}{2.25} ) + 10.96 \cdot ( \frac{P 3 - 0.445}{6.355} ) + 16.122 \cdot ( \frac{Pp - 1506}{566} ) + ( \frac{P 0 - 23}{23} ) \cdot ( ( \frac{P 0 - 23}{23} ) \cdot ( - 18.349 ) + ( \frac{P 0 - 23}{23} ) \cdot ( ( \frac{Pp - 1506}{566} ) \cdot ( - 9.097 ) )$$

$$P 0 , P 2 and P 3 of substances in category B \qquad Equation 2$$

$$Substance absorption [ \% ] = 65.750 + 26.303 \cdot ( \frac{P 0 - 23}{23} ) + 25.491 \cdot ( \frac{P 2 - 2.25}{2.25} ) + 13.729 \cdot ( \frac{P 3 - 0.445}{6.335} ) + 4.355 \cdot ( \frac{Pp - 324.5}{196.5} ) + ( \frac{P 0 - 23}{23} ) \cdot ( ( \frac{P 0 - 23}{23} ) \cdot ( - 21.011 ) )$$

$$P 0 , P 2 and P 3 of substances in category C \qquad Equation 3$$

$$Substance absorption [ \% ] = 44.685 + 18.211 \cdot ( \frac{P 0 - 23}{23} ) + 7.359 \cdot ( \frac{P 2 - 2.25}{2.25} ) + 13.704 \cdot ( \frac{P 3 - 0.445}{6.335} ) - 17.023 \cdot ( \frac{Pp + 48}{80} ) + ( \frac{P 0 - 23}{23} ) \cdot ( ( \frac{P 0 - 23}{23} ) \cdot ( - 8.837 ) ) + ( \frac{P 2 - 2.25}{2.25} ) \cdot ( ( \frac{P 2 - 2.25}{2.25} ) \cdot 20.796 ) + ( \frac{( P 2 - 2.25 )}{2.25} ) \cdot ( ( \frac{( Pp + 48 )}{80} ) \cdot 11.267 ) + ( \frac{( Pp + 48 )}{80} ) \cdot ( ( \frac{( Pp + 48 )}{80} ) \cdot ( - 9.363 ) )$$

$$P 0 , P 2 and P 3 of substances in category D \qquad Equation 4$$

$$Substance absorption [ \% ] = 4.536 + 7.312 \cdot ( \frac{P 0 - 23}{23} ) + 0.53 \cdot ( \frac{P 2 - 2.25}{2.25} ) + 8.132 \cdot ( \frac{P 3 - 0.445}{6.355} ) + 3.109 \cdot ( \frac{Pp + 484.5}{330.5} ) + ( \frac{P 3 - 0.445}{6.355} ) \cdot ( ( \frac{P 3 - 0.445}{6.355} ) \cdot 24.302 ) + ( \frac{P 0 - 23}{23} ) \cdot ( ( \frac{Pp + 484.5}{330.5} ) \cdot 7.485 ) + ( \frac{( P 2 - 2.25 )}{2.25} ) \cdot ( ( \frac{( Pp + 484.5 )}{330.5} ) \cdot 6.526 )$$

$$P 0 , P 2 and P 3 of substances in categories A - D \qquad Equation 5$$

$$Substance absorption [ \% ] = 61.777 + 19.849 \cdot ( \frac{P 0 - 23}{23} ) + 19.715 \cdot ( \frac{P 2 - 2.25}{2.25} ) + 12.549 \cdot ( \frac{P 3 - 0.445}{6.355} ) + 31.173 \cdot ( \frac{Pp - 628.5}{1443.5} ) + ( \frac{P 0 - 23}{23} ) \cdot ( ( \frac{P 0 - 23}{23} ) \cdot ( - 15.276 ) ) + ( \frac{( Pp - 628.5 )}{1443.5} ) \cdot ( ( \frac{( Pp - 628.5 )}{1443.5} ) \cdot ( - 13.77 ) )$$

$$P 1 , P 2 and P 3 of substances of category A \qquad Equation 6$$

$$Substance absorption [ \% ] = 106.35 + 17.018 \cdot ( \frac{P 1 - 36}{34} ) + 5.683 \cdot ( \frac{P 2 - 2.7}{1.8} ) + 8.715 \cdot ( \frac{P 3 - 1.51}{5.29} ) + 13.738 \cdot ( \frac{Pp - 1506}{566} ) \cdot ( - 7.881 ) ) + ( \frac{( P 3 - 1.51 )}{5.29} ) \cdot ( ( \frac{( Pp - 1506 )}{566} ) \cdot ( - 6.358 ) )$$

$$P 1 , P 2 and P 3 of substances of category B \qquad Equation 7$$

$$Substance absorption [ \% ] = 103.605 + 23.597 \cdot ( \frac{P 1 - 36}{34} ) + 8.492 \cdot ( \frac{P 2 - 2.7}{1.8} ) + 13.131 \cdot ( \frac{P 3 - 1.51}{5.29} ) + 4.355 \cdot ( \frac{Pp - 342.5}{196.5} ) + ( \frac{P 1 - 36}{34} ) \cdot ( ( \frac{P 1 - 36}{34} ) \cdot ( - 47.237 ) )$$

$$P 1 , P 2 and P 3 of substances of category C \qquad Equation 8$$

$$Substance absorption [ \% ] = 67.102 + 16.546 \cdot ( \frac{P 1 - 36}{34} ) + 5.095 \cdot ( \frac{P 2 - 2.7}{1.8} ) + 12.879 \cdot ( \frac{P 3 - 1.51}{5.29} ) - 14.769 \cdot ( \frac{Pp + 48}{80} ) + ( \frac{P 1 - 36}{34} ) \cdot ( ( \frac{P 1 - 36}{34} ) \cdot ( - 26.07 ) ) + ( \frac{P 2 - 2.7}{1.8} ) \cdot ( ( \frac{P 2 - 2.7}{1.8} ) \cdot 15.507 ) + ( \frac{( P 2 - 2.7 )}{1.8} ) \cdot ( ( \frac{( Pp + 48 )}{80} ) \cdot 9.008 ) + ( \frac{( Pp + 48 )}{80} ) \cdot ( ( \frac{( Pp + 48 )}{80} ) \cdot ( - 9.363 ) )$$

$$P 1 , P 2 and P 3 of substances of category D \qquad Equation 9$$

$$Substance absorption [ \% ] = 8.15 + 6.392 \cdot ( \frac{P 1 - 36}{34} ) - 2.386 \cdot ( \frac{P 2 - 2.7}{1.8} ) + 14.656 \cdot ( \frac{P 3 - 1.51}{5.29} ) - 6.864 \cdot ( \frac{Pp + 484.5}{330.5} ) + ( \frac{P 3 - 1.51}{5.29} ) \cdot ( ( \frac{P 3 - 1.51}{5.29} ) \cdot 16.922 ) + ( \frac{P 1 - 36}{34} ) \cdot ( ( \frac{Pp + 484.5}{330.5} ) \cdot 8.672 )$$

$$P 1 , P 2 and P 3 of substances of category A - D \qquad Equation 10$$

$$Substance absorption [ \% ] = 85.514 + 18.241 \cdot ( \frac{P 1 - 36}{34} ) + 7.315 \cdot ( \frac{P 2 - 2.7}{1.8} ) + 11.097 \cdot ( \frac{P 3 - 1.51}{5.29} ) + 31.174 \cdot ( \frac{Pp - 628.5}{1443.5} ) + ( \frac{P 1 - 36}{34} ) \cdot ( ( \frac{P 1 - 36}{34} ) \cdot ( - 34.366 ) ) + ( \frac{P 2 - 2.7}{1.8} ) \cdot ( ( \frac{P 2 - 2.7}{1.8} ) \cdot 8.046 ) + ( \frac{Pp - 628.5}{1443.5} ) \cdot ( ( \frac{Pp - 628.5}{1443.5} ) \cdot ( - 13.771 ) )$$

Release of the Growth Factor VEGF165 from the Hydrogels

The hydrogel materials are prepared in accordance with the mixtures predetermined in Tables 5-1 to 5-5. 10 μl of the precursor solution with 100 ng of recombinant human VEGF165 (38.2 kDa; Peprotech, Germany) were mixed in a small tube with low protein binding and at the same time crosslinked. The hydrogel materials are placed in 300 μl serum-free Dulbecco's Modified Eagle Medium (DMEM; Gibco Life Technologies, USA) with 0.1% BSA and 0.05% ProClin300 at RT and after 3, 6, 24, 96, 168 and 240 h the medium is removed, collected and stored at −80° C. and replaced by fresh medium. The amount of protein VEGF released is quantified for each sampling time using enzyme-linked immunosorbent assay (ELISA; DuoSet kit, R&D Systems, USA) according to the manufacturer's protocol.

The influence of parameters P0, P1, P2 and P3 of the anionically charged hydrogel materials on the long-term release of the growth factor VEGF165 from the respective hydrogel material was analyzed over a period of 240 hours (10 days) (see Table 7).

Similar to the hydrogel materials used in the binding experiments, VEGF165 was released from hydrogel materials with similar mechanical properties (storage module in the range of 2-4 kPa), so that the mesh size was large enough to allow free protein transport. The selected hydrogel material properties were designed such that they had a comparably high concentration of the anionically charged groups (not building blocks), expressed by parameter P0 in the range of 35-63 μmol/ml and strongly variable integral sulfate concentrations (P1), local sulf(on)ate densities (P2) and amphiphilia of the anionic building blocks (P3). All hydrogel materials showed an initial rapid release of the VEGF165 within the first 24 h, followed by nearly constant, slow, linear release profiles over 240 h. Due to the absence of anionically charged building blocks, PEG/PEG hydrogel materials showed the highest VEGF165 release with 55% release at the end of the ten-day study.

Tables 8-1 to 8-2 show a cumulative release of VEGF165 as a bioactive substance from hydrogel materials of types 53 to 62. (mean±standard deviation, n=6), top row: Hydrogel material types, rows 2 to 5: Parameters P0, P1, P2, P3, rows 7-12: Substance release values in % at 3, 24, 48, 96, 168 and 240 h.

The highest VEGF165 release for the anionic hydrogel materials was achieved for GB2-UGB3 type 54 with 32.8% protein release, followed by GB4-UGB3 type 56 E50 with a 26.9% release after 240 hours (see Table 7). The results show that inversely to the binding, a low parameter P2 (local charge density, decisive for specific charge interactions) and a low parameter P3 (and therefore hydrophilic anionic building block) intensifies the release of VEGF165.

### Immune Cell Migration Depending on the Graded Sequestration of the Substance IL-8

The 1:1 mixed precursor solutions (see Tables 5-1 to 5-5, GB2-UGB3 63 to UGB1-UGB3 66) are pipetted on the bottom of a 24 well plate and flattened with Teflon film in order to create a thin gel layer covering the bottom of the well. The hydrogel layers are sterilized by UV irradiation for 20 minutes under the sterile bench and incubated with sterile 1% BSA for one hour and then washed with PBS. The hydrogels are then incubated for 24 h in 850 μl of IL-8 solution (0, 10 or 100 ng/ml of IL-8 in RPMI 1640 medium (Gibco, Life Technologies, USA) with 0.1% BSA). Fresh whole blood is taken from healthy human volunteers and the blood is anticoagulated with hirudin (Refludan 1 μM (Celgene Munich, Germany)). 200 μl of blood are now introduced into suspended Transwell inserts (8 μm, PET, Merck Millipore) with the hydrogel layer and the IL-8 solution in the bottom and incubated for 2 h at 37° C. The immigrant cells are removed from the wells and washed with RPMI. For flow cytometry, the cells are stained with PE-labeled anti-human CD15 antibodies (BioLegend, USA) for 30 minutes in the dark at room temperature. For each hydrogel condition, the amount of CD15+ migrating granulocytes is normalized to a PEG hydrogel incubated with RPMI (-ve control).

A strategy for the treatment of excessive inflammation resulting from multilayered processes consists in disturbing effector molecules, such as chemokines. During an inflammation, chemokines selectively recruit and activate immune cells to detect and combat pathogens and injuries. However, the overexpression of chemokines can lead to an uncontrolled influx of immune cells, which drives the production of further inflammatory mediators, including even more chemokines, and finally leads to long-lasting or chronic inflammation. Interleukin-8 (IL-8) is responsible for the recruitment of immune cells at infection sites, contributes to the transendothelial migration of immune cells and is involved in many inflammatory diseases (Gerard et al., 2001, doi: 10.1038/84209) and chronic wounds (Kroeze et al.,2012 doi: 10.1111/j.1524-475X.2012.00789.x).

In order to evaluate a biologically relevant application of the parameter-dependent sequestration of chemokines by means of anionically charged hydrogel materials, the chemoattractive function of IL-8 was investigated in realistic transmigration assays using human whole blood as a biofluid, in which special immune cells, so-called granulocytes, are known to react to the IL-8 activation by chemotaxis during inflammation (REF). The addition of IL-8 in culture medium, preincubated with thin hydrogel films of UGB2 and GB2 or GB7 (types 63 and 64), showed the effectiveness of these hydrogels for the sequestration of IL-8, which was demonstrated by the minimized transmigration of granulocytes (see Tables 8-1 to 8-2). At two biologically relevant concentrations of IL-8 (10 and 100 ng/ml), these hydrogels showed a significant inhibition of granulocyte migration activity (significance value p<0.0001) compared to their UGB1/UGB3 positive control hydrogels (type 66), see Tables 8-1 to 8-2. In fact, the number of migrating granulocytes in these two types of hydrogel material was as low as in the UGB1/UGB2 hydrogel, which was not exposed to IL-8 (negative control), which indicates an unprecedented capacity for collecting and retaining the IL 8 within the hydrogel over a high IL-8 concentration range.

In contrast, GB9 hydrogel materials reduced granulocyte migration only slightly (but not significantly) at 10 ng/ml and no migration inhibition was indicated at 100 ng/ml. Moreover, none of the other hydrogel materials based on anionically charged building blocks showed a significant influence on the migration behavior of the granulocytes. The chemokine-intercepting components GB7 and GB2 within the sterically accessible hydrogel network showed superior IL-8 binding and granulocyte inhibition, not only because of the high anionic charge density (global P1 and local P2) within the network, but also because of the ability to not only electrostatically stabilize the interaction of the protein by hydrophobic interactions, similar to the IL 8 interaction with its receptor (IL-8r1) (Clubb et al., 1994 doi: 10.1016/0014-5793(94)80123-1). These findings are consistent with the predictive values of regression analysis.

Tables 8-1 to 8-2 show a migration of immune cells from human whole blood and the reaction of blood granulocytes to an IL-8 solution which was incubated with various anionically charged building blocks, relative to the number of cells without IL-8 which migrated into UGB1/UGB3 negative control hydrogel materials. (mean±standard deviation, n=5).

### Cell Culture: HUVEC Morphogenesis

Human umbilical vein endothelial cells (HUVEC) are isolated as described above (Weis et al., 1991 doi: 10.1016/0049-3848(91)90245-r) and cultivated in endothelial cell growth medium (ECGM; Promocell, Germany), which contains the supplement mix (Promocell, Germany) and 1% Pen-Strep (Sigma-Aldrich, Germany) in fibronectin-coated 75 cm2 culture bottles in a moistened incubator at 5% CO2 and 37° C. After reaching 80% confluence, the cells are dissolved with 0.5% trypsin-EDTA solution (Sigma-Aldrich, Germany), collected, centrifuged at 1000 rpm and sown again in a suitable density until further use. For all experiments, cells of the passage 2-6 are used. The HUVEC cell culture experiments are performed according to Limasale et al. 2020 (Limasale, et al. , 2020 doi: 10.1002/adfm.202000068). For experimental evaluation, the HUVEC are stained with the cytosolic tracer carboxyfluorescein diacetate, succinimidylester (CFDA-SE) (Vybrant® CFDASE Cell Tracer Kit, Thermo Fischer Scientific, USA) according to the manufacturer's protocol before they are embedded in the hydrogel.

The crosslinker UGB3 and the maleimized, charged building blocks GB1-GB9 are dissolved in HUVEC culture medium (Tables 5-1 to 5-5, GB7-UGB3 67 to UGB1-UGB3 699) and then the adhesive peptide CWGGRGDSP (cRGD, 990 g/mol) is added in a molar ratio of 2:1 to the charged building block. The polymer-RGD mixture is then functionalized with VEGF165 (PeproTech, USA) at a final concentration of 20 μg/ml and the same volume of HUVEC suspension is added. With this solution and UGB3 in a volume ratio of 1:1, 20 μl of hydrogel material drops are pipetted onto hydrophobic p microscope slides as 8-well chambers (Ibidi, Germany). After in-situ crosslinking, the gels are immediately placed in the cell culture medium and, on the third day, are examined with a confocal DragonFly spinning disk microscope (Andor, Oxford Instruments, United Kingdom). In order to quantify the extent of the formation of tubular structures in 3D, 100 μm thick z-layers were analyzed with the Imaris software (version 9.2.1, Bitplane AG, Switzerland) using the filament tracer module. A threshold algorithm is used to create a 3D image of the tubular structures from the CFDA-SE cell coloring. The total area of the vessels is calculated from these images.

The results of the HUVEC cell culture experiments are represented in Table 9. Hydrogel materials without anionically charged building blocks (UGB1-UGB3 69) showed minimal cell expansion. The total area of the tubular structures was higher in hydrogels with GB7 (GB7-UGB3 67) and GB9 (GB9-UGB3 68). The synergistic effect of selecting the correct hydrogel material parameters allowed a controlled release of the VEGF165 to stimulate HUVEC morphogenesis and tube formation.

Table 10 shows the cell culture of HUVECs, row 1: Hydrogel type, rows 2-4: Parameters P0 to P3, row 6: Surface of tubular structures as desired cell morphology (mean±standard deviation, n=5).

### Cell Culture: HK-2 Tubulogenesis

The cell culture experiments with HK-2 (human kidney cell-2) are performed on the basis of (Weber et al., 2017 doi: 10.1016/j.actbio.2017.05.035). The HK-2 cells (American Type Culture Collection, ATCC, #CRL-2190) are cultivated in DMEM/F-12 medium (Gibco, Life Technology, USA) with 10% fetal bovine serum (FBS, Biochrom, Germany) and 1% Pen-Strep (Sigma-Aldrich, Germany). The medium for the 2D and 3D cultures is changed every other day. The cells of passages 11-20 are used and 3D cultures are cultivated for 3 weeks.

The hydrogel building blocks are dissolved in PBS with 1% of Pen-Strep, wherein the HK-2 cells (suspended in a quarter of the total volume) are mixed to the maleimized polymer dissolved in a quarter of the total volume (GB3-UGB3 70 to UGB1-UGB3 76). The cell concentration is 2.5*106 cells/ml, which corresponds to 50,000 cells/20 μl gel. The crosslinker UGB3 is dissolved in half of the total volume, mixed in both precursor solutions and 20 μl of hydrogel drops on 6 mm glass panes are pipetted with SigmaCote®.

For immunohistochemistry, the samples are first fixed for 20 minutes in 4% paraformaldehyde, washed 2× with PBS and then immunostained. The samples are blocked with 0.1% BSA in PBS with 0.5% Triton X-100 for 1 h and incubated overnight with primary antibodies (mouse anti-β-catenin, 1:100, BD Transduction Laboratories, USA) at 4° C. After washing three times with PBS/0.1% Triton X 100/0.1% BSA, secondary antibodies (Alexa Flour 488 Goat-anti-mouse, 1:200, Invitrogen, USA) are added and incubated at RT for 1 h. For the actin and lotus tetragonobolus lectin (LTL) staining, phalloidin atto 550 (1:50, Sigma-Aldrich, Germany) or fluorescein-labeled LTL (1:500, Vector Laboratories, USA) is added during incubation with the secondary antibodies. The samples are then washed and incubated with the dye DRAQ5 (Thermo Fisher Scientific, USA) for 20-30 minutes. The fluorescence images are recorded with the confocal DragonFly Spinning Disk microscope.

To quantify the number and length of the tube structures formed, single-phase 4× EDF contrast images are taken of each hydrogel after fixing the cells. All cell groups on the recorded images are counted with the Image J software. The colonies are categorized into round spheroids, spiky spheroids or tubular structures. They are considered tubular if their length to diameter ratio is greater than 5. If a colony has several protuberances/tubular protuberances, the longest is evaluated for each colony, the rest is neglected. Since the diameter varies along the tubules, two to three regions are measured and averaged. The number of tubular structures formed is determined as a percentage of the colonies forming tubules. The absolute tube length of all tubular colonies is evaluated with the Image J software.

In order to demonstrate possible applications of hydrogels based on anionically ionizable building blocks in three-dimensional in vitro tissue models, HK-2 cells were cultivated in 3D as a human kidney tubulogenesis model. HK-2 cells are a human renal proximal tubule cell line which can form polarized tubes under favorable matrix conditions. We have previously reported starPEG hep hydrogels as a robust, adjustable matrix that stimulates proximal renal tubulogenesis and forms tubular structures with the same morphology and architecture, comparable to human tubules in vivo (Weber et al., 2017 doi: 10.1016/j.actbio.2017.05.035).

To investigate and optimize the hydrogel material properties based on the anionically charged building blocks for kidney tubulogenesis, the three polymers GB7, GB3 and GB4 were selected. The influence of different concentrations of the polymers (2, 20 and 100%) within the maleimized building blocks of the hydrogel material was also tested. The various anionically charged building blocks were crosslinked with UGB3 in order to allow a cell-responsive restructuring of the matrix via cell-secreted enzymes, the so-called matrix metallopreteinases (MMPs). The degradable hydrogel materials were adjusted so that they have a similar stiffness of 500 Pa. As already reported (Weber et al., 2017 doi: 10.1016/j.actbio.2017.05.035), GB9-UGB3 hydrogels were used as a positive control, while the non-sulfated UGB1 was used with UGB3 hydrogels as a negative control. The colonies were evaluated with regard to the measure of tubulogenesis and average tube length by counting the colonies and categorizing them according to their stage of tubulogenesis (round spheroids, spheroids with extensions (“spiky spheroids”) or tubular structures). The colonies were classified as tubular structures if their length to diameter ratio was 5:1 or higher.

On average, GB9 hydrogel materials showed 13±7% of tubular colonies, while UGB1-UGB3 hydrogels did not promote tubulogenesis as was expected and no tubular colonies were observed. Compared with GB9 hydrogels, GB7 (with 2% of the maleimized charged building blocks, GB7-UGB1-UGB3 74) and GB3 (with 100%, GB3-UGB3 70) showed the tubular colonies of 19±8% and 14±11%, respectively. Interestingly, a higher GB7 concentration than 2% within the hydrogel materials leads to the apoptosis of HK-2 cells, which indicates that at higher concentrations the GB7 has a high affinity for essential proteins in the culture media which capture the proteins and thus extract vital nutrients from the cells. The highly sulfonated analog of GB3 is GB4, whose hydrogel materials exhibited only a low tubule genesis both at 100% (GB4-UGB3 72) and at 20% concentration (GB4-UGB1-UGB373) (4±7% and 2±3%, respectively), which indicates the importance of adjusting the charge of the matrix in order to control renal tubule genesis. Some diseases even had structures similar to the morphology of the proximal tubule in vivo, wherein the HK-2 cells formed spheroids with lumens that expressed β-catenin along the basolateral membranes. Under the most promising matrix conditions, GB9 (GB9-UGB3 75)-GB7 2% (GB7-UGB1-UGB3 74)- and GB3 100% (GB3-UGB3 70) hydrogel materials, the average tube length was comparable to 140, 176 and 173 μm, respectively. The tubular structures of the anionically ionizable hydrogel material bound both to the proximal tubule-specific lotus tetragonolobus lectin (LTL) as an apical marker and to β-catenin as basolateral marker and thus confirmed that the tubular structures in the hydrogels retained their differentiated phenotype and their polarization in the reported 3D culture. In summary, it can be said that the anionically charged hydrogels offered a favorable matrix for kidney tubulogenesis, comparable to the well-established heparin-based hydrogel model, and produced polarized tubule structures with similar morphology and architecture.

Table 11 shows results of a cell culture of HK-2 cells, row 1: Hydrogel material type, rows 2-4: Parameters P0 to P3, row 6: Round spheroids in %, row 7: Spiky spheroids in %, row 8: Tubular structures in % (mean±standard deviation, n=5).

### Statistical Analysis

All statistics were performed with GraphPad Prism 5 (GraphPad Software Inc.). All values are indicated as a mean±standard deviation for at least three independent samples. If specified, the statistical analysis was performed with a one-sided variance analysis (ANOVA) followed by a Tukey post-hoc test with a confidence interval of 95%.

1.-24. (canceled)

25. A configurable hydrogel material comprising,
a hydrogel based on at least three nucleophilic groups carrying anionically charged building blocks under physiological conditions, and un-charged building blocks which have at least two electrophilic groups for reaction with the nucleophilic groups,
said charged and uncharged building blocks are crosslinked to a polymer network by a reaction of the electrophilic and nucleophilic groups,
wherein the resulted hydrogel material is configured on the basis of parameters, P0, P1, P2, P3 defining the anionic building blocks,
said parameter P0 corresponding to the number of ionized anionic groups, assuming a 30% ionization of all ionic groups, per unit value of the hydrogel material swollen under physiological conditions,
said parameter P1 corresponding to a value from the number of strongly anionic groups, having an intrinsic pKs value of less than 2.5 per unit volume of the hydrogel material swollen under physiological conditions,
said parameter P2 corresponding to a value from the number of strongly anionic groups, having an intrinsic pKs value of less than 2.5, per repeat unit divided by the molar mass of the repeating unit, and
said parameter P3 corresponding to a value of amphiphilicity of the anionically charged building blocks, such that the hydrogel material is able to sequester substances into the hydrogel material and able to deplete substances in a biofluid and/or release substances from the hydrogel material into a the biofluid and to deplete the substances in the hydrogel material.

26. A configurable hydrogel material comprising,
charged building blocks selected from the group consisting of poly(acrylic acid-co-4-acrylamidomethylbenzenesulfonic acid), poly(acrylic acid-co-acrylamidoethanesulfonic acid) and poly(acrylic acid-co-acrylamidoethane hydrogen sulfate) and
uncharged building blocks in the form of polymers containing amino or thiol groups or crosslinker molecules having at least two amino or thiol groups,
said charged and uncharged building blocks crosslinked to form a polymer network by activation of carboxyl groups of the charged building blocks with EDC/sulfo-NHS by direct crosslinking with the amino group-containing polymers or the crosslinker molecules with the at least two amino groups, in each case with amide formation, or functionalization of the activated carboxyl groups with bifunctional crosslinker molecules, each containing an amino group and a group capable of Michael-type addition, and subsequent crosslinked with the polymers containing thiol groups or the crosslinker molecules containing the at least two thiol groups, in each case via a Michael-type addition,
said hydrogel material defined on the basis of three building blocks carrying the charged groups, selected from the group of parameters P selected from a group of parameters P0, P1, P2, P3,
said parameter P0 corresponding to a value from the number of ionized anionic groups, assuming a 30% ionization of all anionic groups, per unit volume of the hydrogel material swollen under physiological conditions,
said parameter P1 corresponding to a value from the number of strongly anionic groups, with an intrinsic pKS value smaller than 2.5, per unit volume of the hydrogel material swollen under physiological conditions,
said parameter P2 corresponding to a value of the number of strongly anionic groups, with an intrinsic pKS value lower than 2.5, per repeating unit divided by the molar mass of the repeating unit,
said parameter P3 corresponding to a value describing the amphiphilicity of the anionic charged building blocks such that the hydrogel material is able to sequester substances into the hydrogel material and able to deplete substances in a biofluid and/or release substances from the hydrogel material into a the biofluid and to deplete the substances in the hydrogel material.

27. The configurable hydrogel material of claim 24, wherein the group capable of Michael-type addition is selected from maleimide, vinyl sulfone or acrylate groups.

28. The configurable hydrogel material according to claim 24, wherein the polymers containing amine and thiol groups as uncharged building blocks are selected from the group consisting of polyethylene glycols (PEG), poly(2-oxazolines) (POX), polyvinylpyrrolidones (PVP), polyvinyl alcohols (PVA) and polyarylamides (PAM) wherein the amine or thiol group-containing crosslinker molecules are non-polymeric and bifunctional.

29. The configurable hydrogel material according to claim 26, wherein the charged building block is selected from the group consisting of poly(acrylic acid-co-4-acrylamidomethylbenzenesulfonic acid) having variable molar ratios of acrylic acid to 4-acrylamidomethylbenzenesulfonic acid in the range of 9:1 to 1:9 and molar masses in the range of 5,000 to 100,000 g/mol, poly(acrylic acid-co-acrylamidoethanesulfonic acid) with variable molar ratios of acrylic acid to acrylamidoethanesulfonic acid in the range from 9:1 to 1:9 and molar masses in the range from 5,000 to 100,000 g/mol, and poly(acrylic acid-co-acrylamidoethane hydrogen sulfate) with variable molar ratios of acrylic acid to acrylamidoethane hydrogen sulfate in the range from 9:1 to 1:9 and molar masses in the range from 5,000 to 100,000 g/mol.

30. The configurable hydrogel material according to claim 23, wherein polymers with conjugated enzymatically cleavable peptides having either lysine or cysteine as reactive amino acid in the peptide sequence are used as uncharged building blocks for polymer network formation.

31. The configurable hydrogel material according to claim 28, wherein the enzymatically cleavable peptides are cleavable by human or bacterial proteases selected from the group consisting of MMPs, cathepsins, elastases, aureolysin and blood coagulation enzymes.

32. The configurable hydrogel material according to claim 29, wherein bioactive and/or anti-adhesive molecules having an amino or carboxyl group and/or cell-instructive peptides are attached via lysine or cysteine in the sequence to the charged building blocks poly(acrylic acid-co-4-acrylamidomethylbenzenesulfonic acid) and/or poly(acrylic acid-co-acrylamidoethanesulfonic acid) and/or poly(acrylic acid-co-acrylamidoethane hydrogen sulfate) or their derivatives with groups capable of Michael-type addition, forming a covalent bond to the hydrogel network.

33. The configurable hydrogel material according to claim 30, wherein the bioactive molecules are antibiotics or antiseptics, or pharmaceutical agents.

34. The configurable hydrogel material according to claim 24, wherein the anti-adhesive molecules are polyethylene glycols (PEG) or poly (2-oxazolines) (POX).

35. The configurable hydrogel material according to claim 24, wherein the cell-instructive peptides are peptides collagen, laminin, tenascin, fibronectin and vitronectin derived from structural and functional proteins of the extracellular matrix.

36. The configurable hydrogel material according to claim 24, wherein the bioactive and/or anti-adhesive molecules and/or cell-instructive peptides are covalently coupled to the hydrogel networks via enzymatically cleavable peptide sequences.

37. The configurable hydrogel material of claim 23, wherein the hydrogel material has a storage modulus of 0.2 kPa to 22 kPa.

38. A configurable physically crosslinked hydrogel material based on physical interactions between charged building blocks selected from the group consisting or poly(acrylic acid-co-4-acrylamidomethylbenzenesulfonic acid), poly(acrylic acid-co-acrylamidoethanesulfonic acid) and poly(acrylic acid-co-acrylamidoethane hydrogen sulfate) and uncharged building blocks in the form of polymers with strongly positively charged peptide sequences conjugated to the polymers,
wherein the hydrogel material is configured on the basis of three parameters defining the charged groups bearing building blocks selected from a group of parameters consisting of P0, P1, P2 and P3,
wherein parameter P0 corresponds to a value of the number of ionized, anionic groups, assuming a 30% ionization of all anionic groups, per volume unit of the hydrogel material swollen under physiological conditions, parameter P1 corresponds to a value from the number of strongly anionic groups, with an intrinsic pKₛ value smaller than 2.5, per volume unit of the hydrogel material swollen under physiological conditions, parameter P2 corresponds to a value from the number of strongly anionic groups, with an intrinsic pKₛ value smaller than 2.5, per repeating unit divided by the molar mass of the repeating unit, and parameter P3 corresponds to a value for describing the amphiphilicity of the molecular structure surrounding the anionic groups.

39. The configurable physically crosslinked hydrogel material of claim 36, wherein the strongly positively charged peptide sequences comprise at least ten repeats of lysine or arginine or at least five repeats of dipeptide motifs comprising lysine and alanine or arginine and alanine.

40. A method for determining and providing a configuration for a hydrogel material comprising sequestering bioactive substances in the hydrogel material and depleting the substances in a biofluid and/or releasing substances from the hydrogel material into the biofluid and depleting the substances in the hydrogel material, using a hydrogel material according to claim 23,
wherein substances are classified into at least two categories according to a value PP calculated from the ratio of the net charge of a substance and the water-accessible surface area of the substance,—for each category, for at least two different values of a parameter of a predetermined hydrogel configuration, in each case a substance uptake value is/are experimentally determined on the basis of a percentage substance uptake of a test substance assigned to the category into the hydrogel and/or a substance release value is/are experimentally determined on the basis of a percentage substance release of the test substance from the hydrogel into the biofluid, and a category-specific function is formed in each case on the basis of at least two experimentally determined substance uptake values and/or on the basis of at least two experimentally determined substance release values, on the basis of which further substance uptake values and/or substance release values of further predetermined hydrogel configurations with predetermined parameters are determined, those hydrogel configurations of the hydrogel with predetermined values for the parameters being selected as suitable for influencing the concentration of any substance assignable to a category in the biofluid, for which a category-specific regression function formed from the experimentally determined substance uptake values and the determined substance uptake values and/or the experimentally determined substance release values and the calculated substance release values has a coefficient of determination R² in a range of at least 0.6 to at least 0.7.

41. The method according to claim 38, wherein the predetermined hydrogel configuration is formed with the parameters P0, P2, P3 or P1, P2, P3.

42. The method according to one of claims 38, wherein substances are classified into four categories A, B, C and D on the basis of their PP value, wherein of category A substances with a PP value greater than 940, of category B substances with a PP value in a range from 940 to 128, category C substances with a PP value in the range from 128 to −128, and category D comprises substances with a PP value of less than −128.

43. The method according to any one of claim 38, wherein for the experimental determination of the substance uptake values and/or the substance release values, a value in a range from 0 to 80 μmol/ml is predetermined for the parameter P0, a value in a range from 0 to 150 μmol/ml is predetermined for the parameter P1, a value in a range from 0 to 10 mmol/(g/mol) is predetermined for the parameter P2, and a value in a range from -7*10³ to 7*10³A⁻² is predetermined for the parameter P3.

44. The method according to claim 41, wherein a range of values for at least one parameter P0, P1, P2 or P3 of a hydrogel configuration is determined on the basis of the category-specific regression function for computationally determined substance uptake values and/or substance release values.

45. The method according to claim 42, wherein values of the parameters for the hydrogel configuration P0, P1, P2 and/or for the hydrogel configuration P1, P2, P3 are selected such that in the resulting hydrogel at least one substance of a category is bound in or released from the hydrogel only up to 50% of an initial concentration.

46. A method of using the hydrogel material according to claim 23, for factor management in vivo for controlling angiogenesis, immune diseases, cancers, diabetes, neurodegenerative diseases, Crohn's disease, ulcerative colitis, multiple sclerosis, asthma, rheumatoid arthritis, or cutaneous wound healing and bone regeneration.

47. The method of using the hydrogel material of claim 23, for targeted purification of proteins from cell lysates of microbial or eukaryotic origin.

48. The method of using of the hydrogel material according to claim 23, for the in vitro cell culture and organ culture of cells selected from the group consisting of induced pluripotent stem cells (iPS-), stem cells and precursor cells not to be assigned iPS, primary cells obtained from patients, immortalized cell lines heart tissue, muscle tissue, kidney tissue, liver tissue and nerve tissue.
